(19) United States
(12) Patent Application Publication
(10) Pub. No.: US 2025/0382572 A1
(43) Pub. Date: Dec. 18, 2025

(54) AUTOMATED METHODS FOR DIFFERENTIATING DOPAMINERGIC NEURONS FROM STEM CELLS

(71) Applicant: Aspen Neuroscience, Inc., San Diego, CA (US)

(72) Inventors: David TASTAD, San Diego, CA (US); Louisa ZEBROWSKI, San Diego, CA (US); Branden CLARK, San Diego, CA (US); Antonio FERNANDEZPEREZ, San Diego, CA (US); Bryan CAWTHON, San Diego, CA (US)

(73) Assignee: Aspen Neuroscience, Inc., San Diego, CA (US)

(21) Appl. No.: 19/238,348

(22) Filed: Jun. 13, 2025

Related U.S. Application Data
(60) Provisional application No. 63/659,765, filed on Jun. 13, 2024.

(51) Int. Cl. C12N 5/0797; C12M 1/00; C12M 1/36

(52) U.S. Cl. CPC C12N 5/0623; C12M 29/00; C12M 41/48; C12N 2500/38; C12N 2500/40; C12N 2501/13; C12N 2501/15; C12N 2501/155; C12N 2501/16; C12N 2501/41; C12N 2501/727; C12N 2506/45

(57) ABSTRACT

The present disclosure provides automated methods of differentiating pluripotent stem cells, including induced pluripotent stem cells, into lineage-specific floor plate midbrain progenitor cells, such as dopaminergic neuronal progenitor cells, determined dopaminergic neuronal progenitor cells, committed dopaminergic neuronal progenitor cells and/or dopaminergic neuronal cells. Also provided are compositions uses thereof, such as for treating neurodegenerative diseases and conditions, including Parkinson's disease, and articles of manufacture and kits for use thereof.

### CROSS REFERENCE TO RELATED APPLICATIONS

This application claims priority to U.S. Provisional Application No. 63/659,765, filed Jun. 13, 2024, entitled “AUTOMATED METHODS FOR DIFFERENTIATING DOPAMINERGIC NEURONS FROM STEM CELLS,” the contents of which are incorporated herein by reference in its entirety for all purposes.

### BACKGROUND OF THE INVENTION

### Field

The present disclosure relates to methods of differentiating pluripotent stem cells, including induced pluripotent stem cells, into lineage-specific floor plate midbrain progenitor cells, determined dopaminergic neuronal progenitor cells, committed dopaminergic neuronal progenitor cells and/or dopaminergic neuronal cells. Also provided are compositions of the differentiated cells and therapeutic uses thereof, such as for treating neurodegenerative conditions and diseases, including Parkinson's disease, and articles of manufacture and kits thereof.

### Background

Parkinson's Disease (PD) is a progressive neurodegenerative disorder that leads to debilitating motor complications. Currently, no restorative treatments are available. PD is second only to Alzheimer's disease in prevalence among neurodegenerative disorder, affecting about 0.3% of the general population and 1-2% of the population over age 65. The prevalence of PD is expected to double or triple as the developed world population ages. (Cha et al. (2023) J. Mov. Disord. 16: 22-41; Rong et al. (2021) Neurology 97: e1986-e1993; Dorsey and Bloem (2018) JAMA Neurol. 75:9-10; de Lau and Breteler (2006) Lancet Neurol. 5: 525-535).

A hallmark of PD is dopamine deficiency caused by the progressive loss of dopaminergic neurons in the substantia nigra. By the time of diagnosis, patients typically exhibit substantial nigrostriatal degeneration. Current treatments, such as dopamine replacement therapy (e.g., L-dopa or dopamine agonists), provide symptomatic relief for some patients but are limited by side effects and diminishing efficacy over time. (Cha et al., Weiss et al. (1971) Lancet. 1:1016-1017; Kang and Fahn (1988) Ration. Drug Ther. 22: 1-7).

Cell replacement therapies aimed at restoring lost dopaminergic neurons have been under investigation for decades. One early approach involved transplanting fetal midbrain dopaminergic neurons, with over 300 patients treated worldwide. (Brundin et al., Prog. Brain Res. (2010) 184:265-94; Lindvall, & Kokaia, J. Clin. Invest (2010) 120:29-40). While some patients showed long-term survival and function of transplanted neurons, outcomes were inconsistent. A subset of patients developed graft-induced dyskinesias, likely due to serotonin (5-HT) production by the transplanted fetal cells. (Politis et al., Mov. Disord. (2011) 26: 1997-2003). Additional limitations of fetal tissue transplantation include limited availability and quality of donor tissue, ethical and logistical challenges, and the heterogeneous and poorly defined nature of the grafts. (Mendez et al., Nature Med. (2008); Kordower et al., N. Engl. J. Med. (1995) 332:1118-24; and Piccini et al., Nature Neuroscience (1999) 2:1137-40). Hypotheses for the limited efficacy include insufficient cell numbers, inappropriate developmental stage, and host inflammatory responses. (Bjorklund et al., Lancet Neurol. (2003) 2:437-45).

An alternative strategy involves using pluripotent stem cells (PSCs), including embryonic stem (ES) cells and induced pluripotent stem cells (iPSCs), as a renewable source for regenerative therapies. ES cells, derived from the inner cell mass of preimplantation embryos, can self-renew indefinitely and differentiate into all cell types. (Romito and Cobellis, Stem Cells Int. (2016) 2016:9451492). A recent Phase I clinical trial reported the implantation of ES cell-derived dopaminergic neurons into PD patients, showing good tolerability and preliminary motor improvements (Tabar et al. (2025) Nature (https://doi.org/10.1038/s41586-025-08845-y)). However, ES cell use raises ethical concerns and carries risks of tumorigenicity and immune rejection in allogeneic settings.

iPSCs offer a promising alternative by avoiding ethical issues and enabling autologous transplantation, thereby reducing the risk of immune rejection. iPSCs are generated by reprogramming adult somatic cells using factors such as Oct3/4, Sox2, Klf4, and a Myc family member (Yamanaka factors), restoring pluripotency. (Oct 3/4, Sox2, Klf4, and a Myc family member). See, e.g., U.S. Pat. No. 8,530,238.

Despite progress, current differentiation protocols for generating lineage-specific cells from PSCs often yield heterogeneous populations with variable physiological properties and limited in vivo engraftment potential. For neural applications, cells at an intermediate developmental stage—between progenitor and fully differentiated states—may be more suitable for transplantation. Additionally, there is a need to improve the manufacturability of PSC-derived cell products by reducing production time, cost, and variability. The present invention addresses these challenges by providing improved methods for differentiating PSCs into therapeutically relevant cell types and compositions thereof.

### SUMMARY OF THE INVENTION

In some embodiments, the invention provides methods of differentiating pluripotent stem cells into dopaminergic neuronal progenitor cells. In some embodiments, the methods involve: (a) performing a first incubation comprising non-adherently culturing pluripotent stem cells in a cell culture bag under conditions that promote cellular spheroid formation, wherein the first incubation comprises: (i) exposing the pluripotent stem cells to a first medium that comprises an inhibitor of TGF-β/activin-Nodal signaling and an inhibitor of bone morphogenetic protein (BMP) signaling; and (ii) subsequently exposing the resulting partially differentiated cells to a second medium that comprises at least one activator of Sonic Hedgehog (SHH) signaling and an inhibitor of glycogen synthase kinase 3β (GSK3β) signaling; and (b) transferring the spheroids to a second culture vessel and culturing them under adherent conditions to promote differentiation into dopaminergic neuronal progenitor cells. In some embodiments, a pressure (e.g., mechanical or pneumatic) is applied to the cell culture bag during the first incubation. In some embodiments, the cell culture bag has a plurality of wells or recesses (e.g., microwells) on at least one surface of the cell culture bag.

In some embodiments, the cell culture bag has at least one port, and is operably connected to a fluidic system that includes: a) a first pump configured to deliver the second medium from a media bag into the cell culture bag; and b) a second pump configured to remove the first medium from the cell culture bag into a waste bag, wherein the pumps are operated to effect a media exchange. In some embodiments, the second pump is operated to remove all or a portion of the first medium from the cell culture bag prior to operation of the first pump to deliver the second medium into the cell culture bag. In some embodiments, the first pump and the second pump are operated simultaneously during at least a portion of the media exchange cycle. In some embodiments, the media exchange is regulated by a control system that includes: a) a voltage sensor configured to detect medium thickness or volume within the cell culture bag or a holder unit; and b) a computer system executing software instructions to control the operation of the first and second pumps based on input from the voltage sensor. In some embodiments, a media exchange is performed on each of Days 1 through Day 6 of the first incubation.

In some embodiments, the method further involves agitating the cell culture bag after the first incubation to bring the cellular spheroids into suspension and collecting the cellular spheroids.

In some embodiments, the invention provides methods of differentiating pluripotent stem cells into dopaminergic neuronal progenitor cells, wherein the methods involve: (a) exposing the pluripotent stem cells to the first medium that includes the inhibitor of TGF-β/activin-Nodal signaling and the inhibitor of bone morphogenetic protein (BMP) signaling for at least one day (Day 0) in the absence of: x) an activator of Sonic Hedgehog (SHH) signaling, and y) an inhibitor of glycogen synthase kinase 3β (GSK3β) signaling; and (b) starting on the second day (Day 1) of the first incubation, exposing the cells to the second medium that includes the activator of Sonic Hedgehog (SHH) signaling and the inhibitor of glycogen synthase kinase 3β (GSK3β) signaling.

In some embodiments, the second incubation is performed using an automated cell culture system. In some embodiments, the second culture vessel is a plate or a tissue culture flask. In some embodiments, the second culture vessel is a multiwell vessel. In some embodiments, the second culture vessel is compatible with an automated cell culture system. In some embodiments, the second culture vessel is selected based on compatibility with a Mytos automation platform.

In some embodiments, the invention provides methods of differentiating pluripotent stem cells into dopaminergic neuronal progenitor cells, wherein the methods involve: (a) performing a first incubation comprising culturing pluripotent stem cells in a first culture vessel, wherein the first incubation includes: (i) exposing the pluripotent stem cells to a first medium that comprises an inhibitor of TGF-β/activin-Nodal signaling and an inhibitor of bone morphogenetic protein (BMP) signaling; and (ii) exposing the resulting partially differentiated cells to a second medium that comprises at least one activator of Sonic Hedgehog (SHH) signaling and an inhibitor of glycogen synthase kinase 3β (GSK3β) signaling; and (b) performing a second incubation comprising adherently culturing the cells in a second culture vessel under conditions that promote differentiation into dopaminergic neuronal progenitor cells; wherein at least one of the first incubation and the second incubation is performed using an automated cell culture system.

In some embodiments, the second incubation is performed using an automated cell culture system that includes: (a) a first cell culture container configured to receive a sample therein, and a first lid coupled to the first cell culture container, wherein the first lid comprises a first liquid exchange port and a first gas exchange port; b) a first maturation media container comprising a liquid exchange port and containing a first maturation medium that comprises a ROCK inhibitor (ROCKi), an inhibitor of BMP signaling, and an inhibitor of GSK3β signaling; c) a multiport valve comprising a valve actuator and: a first selectable port, wherein the first selectable port is coupled to the first liquid exchange port of the first lid; and a second selectable port coupled to the liquid exchange port of the first maturation media container; d) a fluid pump comprising a pump actuator, wherein the fluid pump is coupled to the valve; and e) a controller configured to actuate the valve actuator and the pump actuator; wherein the controller: i) sends a first valve control signal to actuate the valve to open the first selectable port to place the first maturation media container in fluid communication with the first liquid exchange port of the first cell culture container, and ii) sends a first pump control signal to actuate the fluid pump to move the first maturation media from the first maturation media container to the cell culture container.

In some embodiments, the automated cell culture system further includes: a) a waste container; and b) at least one upstream or downstream valve configured to selectively place the waste container or the first maturation media container in fluid communication with the first liquid exchange port of the first cell culture container; and the method further comprises directing the controller to perform a media exchange by: i) actuating the at least one valve to place the waste container in fluid communication with the first liquid exchange port of the first cell culture container; ii) actuating the fluid pump to move medium from the first cell culture container to the waste container; iii) actuating the at least one valve to place the first maturation media container in fluid communication with the first liquid exchange port of the first cell culture container; and iv) actuating the fluid pump to move the first maturation medium from the first maturation media container to the first cell culture container. In some embodiments, the second maturation media includes an inhibitor of GSK3β signaling, BDNF, GDNF, ascorbic acid, dbcAMP and TGFβ3. In some embodiments, the second media exchange is conducted on Day 4 of the second incubation.

In some embodiments, the automated cell culture system further comprises: a) a second maturation media container comprising a liquid exchange port and containing a second maturation medium; and b) at least one upstream or downstream valve configured to selectively place the second maturation media container in fluid communication with the first liquid exchange port of the first cell culture container; and wherein the controller is further configured to perform a second media exchange by: i) actuating the at least one valve to place the waste container in fluid communication with the first liquid exchange port of the first cell culture container; ii) actuating the fluid pump to move medium from the first cell culture container to the waste container; iii) actuating the at least one valve to place the second maturation media container in fluid communication with the first liquid exchange port of the first cell culture container; and iv) actuating the fluid pump to move the second maturation medium from the second maturation media container to the first cell culture container.

In some embodiments, the invention provides methods in which the first incubation is performed using an automated cell culture system that includes: (a) at least a first cell culture container configured to receive a sample therein, and a first lid coupled to the first cell culture container, wherein the first lid comprises a first liquid exchange port and a first gas exchange port; (b) at least a first induction media container, wherein the first induction media container comprises a liquid exchange port and contains a first induction media that comprises the inhibitor of TGF-β/activin-Nodal signaling and the inhibitor of bone morphogenetic protein (BMP) signaling; (c) a multiport valve that comprises a valve actuator and: (i) a first selectable port, wherein the first selectable port is aseptically coupled to the first liquid exchange port of the first lid; and (ii) a second selectable port, wherein the second selectable port is aseptically coupled to the liquid exchange port of the first induction media container; (d) a fluid pump that comprises a pump actuator, wherein the fluid pump is coupled to the valve; and (e) a controller that is configured to actuate the valve actuator and the pump actuator; wherein the controller: i) sends a first valve control signal to actuate the valve to open the first selectable port to place the first induction media container in fluid communication with the first liquid exchange port of the first cell culture container, and ii) sends a first pump control signal to actuate the fluid pump to move the media from the first induction media container to the cell culture container.

In some embodiments, the invention provides methods of differentiating pluripotent stem cells into dopaminergic neuronal progenitor cells, wherein the methods involve: (a) performing a first incubation comprising non-adherently culturing pluripotent stem cells in a first cell culture bag under conditions to produce a cellular spheroid, wherein the first incubation includes: (i) exposing the pluripotent stem cells to an inhibitor of TGF-β/activin-Nodal signaling and an inhibitor of bone morphogenetic protein (BMP) signaling; and (ii) exposing the pluripotent stem cells to at least one activator of Sonic Hedgehog (SHH) signaling and an inhibitor of glycogen synthase kinase 3β (GSK3β) signaling; and (b) performing a second incubation comprising adherently culturing cells of the spheroid in a second culture vessel under conditions to further differentiate the cells into dopaminergic neuronal progenitor cells; wherein the second incubation is performed using an automated cell culture system that comprises: (a) at least a first cell culture container configured to receive a sample therein, and a first lid coupled to the first cell culture container, wherein the first lid comprises a first liquid exchange port and a first gas exchange port; (b) at least a first maturation media container, wherein the first maturation media container comprises a liquid exchange port and contains a first maturation media that comprises a ROCKi, an inhibitor of BMP signaling, and an inhibitor of GSK3β signaling; (c) a multiport valve that comprises a valve actuator and: (i) a first selectable port, wherein the first selectable port is aseptically coupled to the first liquid exchange port of the first lid; and (ii) a second selectable port, wherein the second selectable port is aseptically coupled to the liquid exchange port of the first maturation media container; (d) a fluid pump that comprises a pump actuator, wherein the fluid pump is coupled to the valve; and (e) a controller that is configured to actuate the valve actuator and the pump actuator; wherein the cells of the spheroid are placed in the first cell culture container and the controller: i) sends a first valve control signal to actuates the valve to open the first selectable port to place the first maturation media container in fluid communication with the first liquid exchange port of the first cell culture container, and ii) sends a first pump control signal to actuate the fluid pump and to move the first maturation media from the first maturation media container to the cell culture container.

In some embodiments, the first incubation includes: (a) exposing the pluripotent stem cells to a first medium that comprises an inhibitor of TGF-β/activin-Nodal signaling and an inhibitor of bone morphogenetic protein (BMP) signaling for at least one day (Day 0) in the absence of: x) an activator of Sonic Hedgehog (SHH) signaling, and y) an inhibitor of glycogen synthase kinase 3β (GSK3β) signaling; and (b) starting on the second day (Day 1) of the first incubation, exposing the pluripotent stem cells to a second medium that comprises an activator of Sonic Hedgehog (SHH) signaling and an inhibitor of glycogen synthase kinase 3β (GSK3β) signaling.

In some embodiments, the invention provides therapeutic compositions that contain dopaminergic neuronal progenitor cells produced using a method that includes: (a) performing a first incubation comprising non-adherently culturing pluripotent stem cells in a first culture vessel under conditions to produce a cellular spheroid, wherein the first incubation comprises: (i) exposing the pluripotent stem cells to a first medium that comprises an inhibitor of TGF-β/activin-Nodal signaling and an inhibitor of bone morphogenetic protein (BMP) signaling for at least one day (Day 0) in the absence of: a) an activator of Sonic Hedgehog (SHH) signaling, and b) an inhibitor of glycogen synthase kinase 3β (GSK3β) signaling; and (ii) starting on the second day (Day 1) of the first incubation, exposing the pluripotent stem cells to a second medium that comprises an activator of Sonic Hedgehog (SHH) signaling and an inhibitor of glycogen synthase kinase 3β (GSK3β) signaling; and (b) performing a second incubation comprising adherently culturing cells of the spheroid in a second culture vessel under conditions to further differentiate the cells into dopaminergic neuronal progenitor cells; wherein either (i) the first incubation is performed using a microwell cell culture bag or (ii) the first incubation and the second incubation is performed using an automated cell culture system.

### BRIEF DESCRIPTION OF THE FIGURES

FIG. 1 shows an exemplary non-adherent protocol for the differentiation of pluripotent stem cells into dopaminergic neuronal progenitor cells, determined dopaminergic neuronal progenitor cells, committed dopaminergic neuronal progenitor cells or dopaminergic neuronal cells.

FIG. 2 shows EN1, GBX2, and NKX2-1 marker expression (counts per million) in Day 7 cells cultured using each of the following methods: 2D adherent culture, 3D Condition 1, 3D Condition 2, 3D Condition 3, and 3D Condition 4.

FIG. 3 shows LMX1A expression on cells cultured using each of the following methods: 2D adherent culture, 3D Condition 1, 3D Condition 2, 3D Condition 3, and 3D Condition 4. The graph quantifies the percentage of LMX1A expressing cells in the cells from three cell lines cultured using each of the following methods: 2D adherent culture, 3D Condition 1, 3D Condition 2, 3D Condition 3, and 3D Condition 4.

FIG. 4 shows flow cytometry measurements of key lineage markers in dopaminergic progenitor cells obtained using manual (non-automated) adherent and suspension culture methods (n=4). Adherent cultured cells were harvested at Day 20, while suspension culture cells were harvested at Day 16. EphB2, a pan-neuronal marker, shows no difference between cells obtained using adherent and suspension culture techniques. FOXA2 expression is characteristic of floorplate lineage in brain development and suspension culture increased expression of FOXA2 compared to adherent culture via flow cytometry pointing towards a more accurate ventral midbrain dopaminergic neuronal cell fate (* p<0.05). PAX6 expression is characteristic of forebrain lineage in brain development, and flow cytometry shows roughly a 10-fold decrease in PAX6 for suspension culture cells compared to adherent culture cells (* p<0.05). Error bars are shown as the standard error of the mean (S.E.M.).

FIG. 5 shows flow cytometry measurements of key lineage markers in dopaminergic progenitor cells obtained using automated adherent and suspension culture methods.

FIG. 6A shows predicted graft size after implantation into a subject brain for neuronal progenitor cells obtained from manual adherent culture and suspension culture. Graft size predictions were estimated across donor backgrounds (n=4). Across all donors, graft sizes were predicted to be larger under suspension culture conditions. When grouped by culture condition, suspension culture was associated with significantly greater predicted graft sizes (*=p<0.05). Error bars are shown as the standard error of the mean (S.E.M.). FIG. 6B shows predicted dopamine release after implantation into a subject brain, by neuronal cells derived from the neuronal progenitor cells obtained using the adherent culture and suspension culture protocols. When grouped by culture condition, there was no significant difference in predicted dopamine release across differentiation conditions (p>0.05). Error bars are shown as the standard error of the mean (S.E.M.).

FIG. 7A shows predicted graft size after implantation into a subject brain for neuronal progenitor cells obtained from automated adherent culture and suspension culture. Predicted graft size was increased approximately 3-fold for suspension culture versus adherent culture protocols. FIG. 7B shows predicted dopamine release after implantation into a subject brain, by neuronal cells derived from the neuronal progenitor cells obtained using the adherent culture and suspension culture protocols.

FIG. 8A presents a graph comparing dopamine concentrations (nM) in the supernatant at baseline and after 30 minutes of KCl stimulation for cells generated using both adherent and suspension (e.g., Wellbag) manual differentiation protocols. Dopamine release increased significantly following KCl treatment in both culture conditions. Data are shown with normalization. Error bars represent the standard error of the mean (S.E.M.); n=4 for all conditions. FIG. 8B shows the fold change of dopamine production for adherent versus suspension culture using a manual process.

FIG. 9 shows the fold change of dopamine production for adherent versus suspension culture using an automated procedure.

FIG. 10A displays raw serotonin (5HT) concentrations (nM) in the supernatant at baseline and after 30 minutes of KCl stimulation, for cells generated using both adherent and suspension (e.g., Wellbag) manual differentiation protocols. Data are shown with normalization. Error bars represent the standard error of the mean (S.E.M.); n=4 for all conditions. FIG. 10B shows the fold change of serotonin production for adherent versus suspension culture using a manual process.

FIG. 11 shows the fold change of serotonin production for adherent versus suspension culture using an automated procedure.

FIG. 12A shows cell viability for dopaminergic neuronal progenitor cells produced using a manual suspension culture protocol compared to those produced using a manual adherent protocol as determined using the “cell count and viability” protocol on a ChemoMetic NucleoCounter® NC-200 (n=15). Cells generated using the suspension culture condition showed a significant increase in percentage of viable cells (** p<0.01). FIG. 12B shows cell viability obtained using automated suspension culture compared to adherent culture differentiation protocols.

FIGS. 13A-13D show expression by DANPCs produced using manual adherent and suspension culture protocols of two “on target” markers FOXA2 (FIG. 13A) and CORIN (FIG. 13B) for which increased expression is indicative of dopaminergic neuronal progenitor cells, and two “off target” markers PITX2 (FIG. 13C) and NKX2.1 (FIG. 13D) for which increased expression is not characteristic of dopaminergic neuronal progenitor cells. The data in this figure thus demonstrate that the cells produced using the manual Day 1 suspension culture differentiation protocol disclosed herein are more characteristic of dopaminergic neuronal progenitor cells than cells produced using a differentiation protocol in which both the first and the second incubations are performed in manual adherent culture.

FIGS. 14A-14E show expression by DANPCs produced using automated adherent and suspension culture protocols of the two “on target” markers FOXA2 (FIG. 14A), CORIN (FIG. 14B), and EN1 (FIG. 14E) for which increased expression is indicative of dopaminergic neuronal progenitor cells, and two “off target” markers PITX2 (FIG. 14C) and NKX2.1 (FIG. 14D) for which increased expression is not characteristic of dopaminergic neuronal progenitor cells.

FIG. 15 shows a schematic diagram of a device and process for exchanging media in a cell culture bag. To replace media that is currently in the cell culture bag with fresh media, pump 2 (P2) is run in the forward direction to transfer the current media to the waste media bag, and pump 1 (P1) is also run in the forward direction to transfer fresh media from the media bag to the cell culture bag.

FIG. 16 shows an overview of an automated system for manufacturing differentiated cells from induced pluripotent stem cells (iPSCs). A Celligent™ cell processing platform from CellX Technologies is used at the iPSC expansion stage of the process. Two different strategies are shown. Strategy A involves performing the first stage of the differentiation protocol with the cells in non-adherent (suspension) culture (3D), as shown in FIG. 1. Strategy B shows an automated protocol in which the entire differentiation process is conducted using adherent culture (2D).

FIG. 17 shows a schematic of an automated differentiation protocol that uses non-adherent (3D) suspension culture for the first seven days of the process, after which the differentiation is continued using adherent (2D) culture. Differentiated cells are harvested and cryopreserved on Day 17.

FIGS. 18A-18C show a comparison of the yield of dopaminergic neuronal progenitor cells (DANPC) obtained from a protocol for differentiating induced pluripotent cells into dopaminergic neuronal progenitor cells in which the non-adherent suspension culture portion of the protocol diagrammed in FIG. 1 was performed in a multiwell cell culture bag (“Automated”) versus performing the suspension culture portion of the differentiation protocol in a multiwell plate (“Manual”)(n=4). Cells generated using the automated culture condition showed a significant increase in cell yield at day 7 (FIG. 18A) while maintaining high viability (FIG. 18B). Cell yield and viability was tested by running the “cell count and viability” protocol on a NucleoCounter NC-200. Cells generated using the automated protocol also produced a significantly higher number of cells on Day 16 after performing the second part of the protocol on a multiwell plate (FIG. 18C). Yield fold change was calculated by taking total cell yield at Day 16 and dividing by initial cells seeded per million to get yield fold change of cells grown throughout the differentiation process.

FIG. 19A shows the fold expansion (FE) of DANPCs when differentiated in Wellbag (Day 13), and adherent (Day 20). The Wellbag suspension culture protocol yielded several times more DANPCs than the adherent protocols. FIG. 19B shows a comparison of the number of culture vessels required to obtain the target DANPCs for manual adherent culture (top) versus automated suspension culture (bottom).

FIGS. 20A-20B show flow cytometry measurements of key lineage markers in dopaminergic progenitor cells between automated and manual culture (n=2). FOXA2 expression is characteristic of floorplate lineage in brain development and automated culture maintains high expression of FOXA2 compared to manual culture via flow cytometry pointing towards an accurate ventral midbrain dopaminergic neuronal cell fate (FIG. 20A). PAX6 expression is characteristic of forebrain lineage in brain development, and flow cytometry shows a decrease in PAX6 for automated culture compared to manual culture (FIG. 20B). Error bars are shown as the standard error of the mean (S.E.M.).

FIG. 21 demonstrates that 3D automated-derived DANPCs can engraft and survive in the rat brain for at least 21 days post-surgery. The figure shows grafts obtained after implantation of DANPCs differentiated using manual suspension culture (Manual 3D), automated suspension culture (Wellbag 3D) or manual adherent culture (Adherent 2D).

### DETAILED DESCRIPTION OF THE INVENTION

The present disclosure relates to methods of automated lineage-specific differentiation of pluripotent stem cells (PSCs), such as embryonic stem (ES) cells or induced pluripotent stem cells (iPSCs). Specifically provided are methods of directing lineage specific differentiation of PSCs or iPSCs into floor plate midbrain progenitor cells, dopaminergic neuronal progenitor cells, determined dopaminergic neuronal progenitor cells (DDPCs), committed dopaminergic neuronal progenitor cells and/or dopaminergic neuronal cells. The differentiated cells made using the methods provided herein are further contemplated for various uses including, but not limited to, use as a therapeutic to reverse disease of, or damage to, a lack of dopamine-producing neurons in a patient. Because Parkinson's disease (PD) symptoms are primarily due to the selective loss of DA neurons in the substantia nigra of the ventral midbrain, PD is considered suitable for cell replacement therapeutic strategies. The automated differentiation methods provided by the present invention provide a more efficient and cost-effective way to manufacture dopaminergic neuronal progenitor cells that are suitable for treating PD and other neurodegenerative diseases. This system offers a sterile environment that minimizes the risk of contamination, thereby ensuring the integrity and viability of the spheroids. The automation of the system reduces manual handling, which not only decreases the potential for human error but also streamlines the cell processing workflow. This results in a consistent and reproducible process that can be pivotal in the clinical production of an autologous cell therapy.

In some embodiments, the provided methods involve performing a first incubation that involves non-adherently culturing pluripotent stem cells in a first culture vessel under conditions to produce a cellular spheroid. The first incubation involves: (a) exposing the pluripotent stem cells to an inhibitor of TGF-β/activin-Nodal signaling and an inhibitor of bone morphogenetic protein (BMP) signaling; and (b) exposing the pluripotent stem cells to at least one activator of Sonic Hedgehog (SHH) signaling and an inhibitor of glycogen synthase kinase 3β (GSK3β) signaling; and performing a second incubation comprising adherently culturing cells of the spheroid in a second culture vessel under conditions to further differentiate the cells into dopaminergic neuronal progenitor cells. In some embodiments, the first culture vessel is a microwell cell culture bag. The use of a cell culture bag in the first incubation provides significant advantages over previous methods for dopaminergic neuronal progenitor cell differentiation. For example, the cell culture bag protocol is more amenable to automation than other methods. Moreover, the use of a cell culture bag can also provide a higher yield of differentiated cells.

In some embodiments, the invention provides methods for differentiating pluripotent stem cells into dopaminergic neuronal progenitor cells and other cell types by performing the second incubation using an automated cell culture system. Use of an automated cell culture system in the differentiation protocol provides advantages over previous methods, including lower cost of production, greater yield, less requirement for human involvement, and greater automatability.

In some embodiments, the invention provides methods for differentiating pluripotent stem cells into dopaminergic neuronal progenitor cells and other cell types by performing both the first incubation and the second incubation using an automated cell culture system.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### A. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker. When referring to a marker in the cell, such as a transcriptional or translational product, the term refers to the presence of detectable transcriptional or translational product, for example, wherein the product is detected at a level substantially above the level detected carrying out the same procedure with a control under otherwise identical conditions and/or at a level substantially similar to that for a cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker. When referring to a marker in the cell, such as a transcriptional or translational product, the term refers to the absence of detectable transcriptional or translational product, for example, wherein the product is not detected at a level substantially above the level detected carrying out the same procedure with a control under otherwise identical conditions, and/or at a level substantially lower than that for cell known to be positive for the marker, and/or at a level substantially similar as compared to that for a cell known to be negative for the marker.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell RNA sequencing (RNAseq) is commonly used to determine the level of expression of a gene. See, e.g., Conesa et al. (2016) Genome Biology 17: 13 (https://doi.org/10.1186/s13059-016-0881-8) for a review of RNAseq methods.

As used herein, the term “stem cell” refers to a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

As used herein, the term “adult stem cell” refers to an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers.

As used herein, the terms “induced pluripotent stem cell,” “iPS” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

As used herein, the term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

As used herein, the term “pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

As used herein, the term “reprogramming” refers to the process of dedifferentiating a non-pluripotent cell into a cell exhibiting pluripotent stem cell characteristics.

The term “differentiated” or “committed” as used herein refers to a cell or cells that have acquired a cell type-specific function.

A “neuronal precursor cell” is a cell that has a tendency to differentiate into a neuronal or glial cell and does not have the pluripotent potential of a stem cell. A neuronal precursor is a cell that is committed to the neuronal or glial lineage and is characterized by expressing one or more marker genes that are specific for the neuronal or glial lineage. The terms “neural” and “neuronal” are used according to their common meaning in the art and can be used interchangeably herein throughout.

A “dopaminergic cell” or a “differentiated dopaminergic cell” as used herein refers to a cell capable of synthesizing the neurotransmitter dopamine. In some embodiments, the dopaminergic cell is an A9 dopaminergic cell. The term “A9 dopaminergic cell” refers to the most densely packed group of dopaminergic cells in the human brain, which are located in the pars compacta of the substantia nigra in the midbrain of healthy, adult humans.

The terms “dopaminergic neuronal progenitor cell” and “determined dopaminergic progenitor cell” as used herein refers to a cell that will differentiate into a dopaminergic neuron and cannot differentiate into a non-dopaminergic cell. A “determined dopaminergic progenitor cell” is a cell able to differentiate into a dopaminergic neuron independently of its environment. A determined dopaminergic progenitor cell may express Foxa2 or Nurr1. A determined dopaminergic progenitor cell, in some embodiments, does not express substantial levels of serotonin.

A “committed dopaminergic progenitor cell,” as used herein, is a dopaminergic neuronal progenitor cell that is at a differentiation state that follows the determined dopaminergic neuronal progenitor cell stage of differentiation.

As used herein, the term “adherent culture vessel” refers to a culture vessel to which a cell may attach via extracellular matrix molecules and the like, and requires the use of an enzyme (e.g., trypsin, dispase, etc.) for detaching cells from the culture vessel. An “adherent culture vessel” is opposed to a culture vessel to which cell attachment is reduced and does not require the use of an enzyme for removing cells from the culture vessel.

As used herein, the term “non-adherent culture vessel” refers to a culture vessel to which cell attachment is reduced or limited, such as for a period of time. A non-adherent culture vessel may contain a low attachment or ultra-low attachment surface, such as may be accomplished by treating the surface with a substance to prevent cell attachment, such as a hydrogel (e.g., a neutrally charged and/or hydrophilic hydrogel) and/or a surfactant (e.g., pluronic acid). A non-adherent culture vessel may contain rounded or concave wells, and/or microwells (e.g., Aggrewells™). In some embodiments, a non-adherent culture vessel is an Aggrewell™ plate. For non-adherent culture vessels, use of an enzyme to remove cells from the culture vessel may not be required.

As used herein, the term “cell culture” may refer to an in vitro population of cells residing outside of an organism. The cell culture can be established from primary cells isolated from a cell bank or animal, or secondary cells that are derived from one of these sources and immortalized for long-term in vitro cultures.

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use, such as in a mammalian subject (e.g., a human). A pharmaceutical composition typically comprises an effective amount of an active agent (e.g., cells) and a carrier, excipient, or diluent. The carrier, excipient, or diluent is typically a pharmaceutically acceptable carrier, excipient or diluent, respectively.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, a “subject” is a mammal, such as a human or other animal, and typically is human.

As used herein, “Day 0” refers to a 24-hour period in which the cells are plated/seeded at the initiation of the 24 hour period of Day 0, and “Day 1” refers to the subsequent day (also a 24 hour period) that begins 24 hours after the initiation of Day 0. Each subsequent day also refers to a 24 hour period, in succession.

### B. Methods for Differentiating Cells

The present invention provides automated methods of differentiating stem cells, including embryonic stem cells and induced pluripotent stem cells (iPSCs) into neuronal cells, such as dopaminergic neuronal progenitor cells. These methods can involve performing a first incubation that includes culturing pluripotent stem cells in a non-adherent culture vessel under conditions to produce a cellular spheroid. The first incubation involves exposing the pluripotent stem cells to at least one inhibitor of TGF-β/activin-Nodal signaling and at least one inhibitor of bone morphogenetic protein (BMP) signaling. The cells are then exposed to at least one activator of Sonic Hedgehog (SHH) signaling and at least one inhibitor of glycogen synthase kinase 3β (GSK3β) signaling. After the first incubation, the cells are subjected to a second incubation under conditions to neurally differentiate the cells. In some embodiments, the first incubation involves non-adherent culturing conditions, e.g., as described in Section B.2, and, the second incubation involves adherent culture conditions, e.g., as described in Sections B.4 and B.5.

### Samples and Cell Preparation

In embodiments of the provided method, pluripotent stem cells are differentiated into floor plate midbrain progenitor cells, determined dopaminergic neuronal progenitor cells, committed dopaminergic neuronal progenitor cells, and/or, dopaminergic neuronal cells. Various sources of pluripotent stem cells can be used in the method, including embryonic stem (ES) cells and induced pluripotent stem cells (iPSCs).

In some aspects, pluripotency refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphologies associated with pluripotent stem cells.

In some embodiments, pluripotent stem cells are induced pluripotent stem cells (iPSCs), artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka (2006) Cell 126: 663-76.

Methods for generating iPSCs are known. For example, mouse iPSCs were reported in 2006 (Takahashi and Yamanaka), and human iPSCs were reported in late 2007 (Takahashi et al. (2007) Cell 131: 861-872 and Yu et al. (2007) Science 318: 1917-1920). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, the PSCs (e.g., iPSCs) are autologous to the subject to be treated, i.e., the PSCs are derived from the same subject to whom the differentiated cells are administered. In some embodiments, non-pluripotent cells (e.g., fibroblasts) derived from patients having Parkinson's disease (PD) are reprogrammed to become iPSCs before differentiation into neural and/or neuronal cells. In some embodiments, fibroblasts may be reprogrammed to iPSCs by transforming fibroblasts with genes (OCT4, SOX2, NANOG, LIN28, and KLF4) cloned into a plasmid (for example, see, Yu et al., Science DOI: 10.1126/science.1172482). In some embodiments, non-pluripotent fibroblasts derived from patients having PD are reprogrammed to become iPSCs before differentiation into determined dopaminergic neuronal progenitor cells, committed dopaminergic neuronal progenitor cells and/or dopaminergic neuronal cells, such as by use of the non-integrating Sendai virus to reprogram the cells (e.g., use of CTS™ CytoTune™-iPS 2.1 Sendai Reprogramming Kit). In some embodiments, the resulting differentiated cells are then administered to the patient from whom they are derived in an autologous stem cell transplant. In some embodiments, the PSCs (e.g., iPSCs) are allogeneic to the subject to be treated, i.e., the PSCs are derived from a different individual than the subject to whom the differentiated cells will be administered. In some embodiments, non-pluripotent cells (e.g., fibroblasts) derived from another individual (e.g., an individual not having a neurodegenerative disorder, such as Parkinson's disease) are reprogrammed to become iPSCs before differentiation into determined dopaminergic neuronal progenitor cells, committed dopaminergic neuronal progenitor cells and/or dopaminergic neuronal cells. In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting differentiated cells are then administered to an individual who is not the same individual from whom the differentiated cells are derived (e.g., allogeneic cell therapy or allogeneic cell transplantation).

In any of the provided embodiments, the PSCs described herein (e.g., allogeneic cells) may be genetically engineered to be hypoimmunogenic. Methods for reducing the immunogenicity are known, and include ablating polymorphic HLA-A/-B/-C and HLA class II molecule expression and introducing the immunomodulatory factors PD-L1, HLA-G, and CD47 into the AAVS1 safe harbor locus in differentiated cells. Han et al. (2019) Proc. Nat'l. Acad. Sci. USA 116(21):10441-46. Thus, in some embodiments, the PSCs described herein are engineered to delete highly polymorphic HLA-A/-B/-C genes and to introduce immunomodulatory factors, such as PD-L1, HLA-G, and/or CD47, into the AAVS1 safe harbor locus.

In some embodiments, PSC (e.g., iPSCs) are cultured in the absence of feeder cells, until they reach 75-90% confluency, at which point they are harvested and further cultured for differentiation (Day 0). In one aspect of the method described herein, once iPSCs reach 75-90% confluence, they are washed in phosphate buffered saline (PBS) and subjected to enzymatic dissociation, such as with Accutase©, until the cells are easily dislodged from the surface of a culture vessel. The dissociated iPSCs are then re-suspended in media for downstream differentiation into determined dopaminergic neuronal progenitor cells, committed dopaminergic neuronal progenitor cells, and/or dopaminergic neuronal cells.

In some embodiments, the PSCs are resuspended in a basal induction media. In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, at the time of seeding (Day 0), the basal induction media is further supplemented with serum replacement, a Rho-associated protein kinase (ROCK) inhibitor, and certain small molecules, e.g., an inhibitor of TGF-β/activin-Nodal signaling, and an inhibitor of BMP signaling, for differentiation. In some embodiments, the PSCs are resuspended in the same media they will be cultured in for at least a portion of the first incubation.

### Modulators of Neuronal Progenitor Cell Differentiation

Suitable differentiation methods for obtaining neuronal progenitor cells are known to those of skill in the art. Such methods can involve manipulating gene expression either directly (e.g., by delivery of a genetic payload into a cell) or indirectly (e.g., by using a variety of pharmacological agents to tilt the differentiation pathway towards a neuronal fate. Telias (2023) Neural Regen. Res. 18: 1273-1274. The dual-SMAD inhibition protocol is an example of the latter method of neurally differentiating pluripotent stem cells to neuronal progenitor cells (Chambers et al. (2009) Nat. Biotechnol. 27: 275-280. This process can involve exposing pluripotent stem cells to (a) an inhibitor of bone morphogenetic protein (BMP) signaling; (b) an inhibitor of TGF-β/activin-Nodal signaling; and (c) at least one activator of Sonic Hedgehog (SHH) signaling. The method can further include exposing the pluripotent stem cells to at least one inhibitor of GSK3β signaling. Methods for neurally differentiating pluripotent stem cells are also described in, for example, US Patent Publication 2023/0059010, entitled METHODS OF DIFFERENTIATING NEURAL CELLS AND RELATED COMPISITIONS AND METHODS OF USE.

Some examples of suitable small molecules for use in the differentiation protocols are provided below.

In some embodiments, the inhibitor of TGF-β/activin-Nodal signaling is a small molecule. In some embodiments, the inhibitor of TGF-β/activin-Nodal signaling is capable of lowering or blocking transforming growth factor beta (TGFβ)/Activin-Nodal signaling. In some embodiments, the inhibitor of TGF-β/activin-Nodal signaling inhibits ALK4, ALK5, ALK7, or combinations thereof. In some embodiments, the inhibitor of TGF-β/activin-Nodal signaling inhibits ALK4, ALK5, and ALK7. In some embodiments, the inhibitor of TGF-β/activin-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activin-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

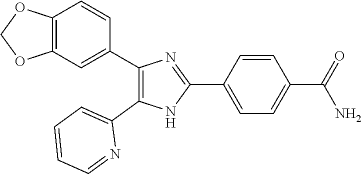 [SMILES: NC(=O)c1ccc(-c2nc(-c3ccc4c(c3)OCO4)c(-c3ccccn3)[nH]2)cc1]

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

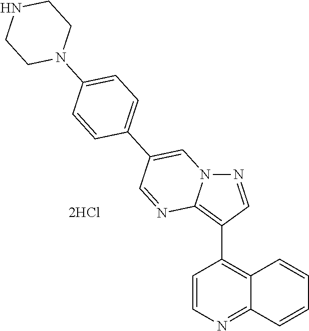 [SMILES: c1ccc2c(-c3cnn4cc(-c5ccc(N6CCNCC6)cc5)cnc34)ccnc2c1]

In some embodiments, the inhibitor of GSK3β signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 33 enzyme. In some embodiments, the inhibitor of GSK3β signaling inhibits GSK3a. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

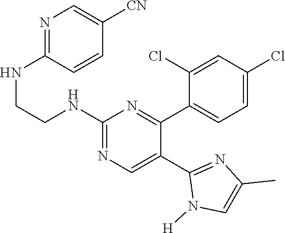 [SMILES: Cc1c[nH]c(-c2cnc(NCCNc3ccc(C#N)cn3)nc2-c2ccc(Cl)cc2Cl)n1]

In some embodiments the media is supplemented with at least one activator of sonic hedgehog (SHH) signaling. SHH refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g., CAS 483367-10-8), having the formula below:

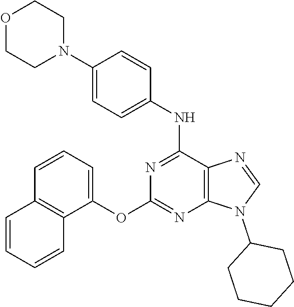 [SMILES: c1ccc2c(Oc3nc(Nc4ccc(N5CCOCC5)cc4)c4ncn(C5CCCCC5)c4n3)cccc2c1]

In some embodiments, the activator of SHH signaling is SHH protein. In some embodiments, the activator of SHH signaling is recombinant SHH protein. In some embodiments, the activator of SHH signaling is recombinant mouse SHH protein. In some embodiments, the activator of SHH signaling is recombinant human SHH protein. In some embodiments, the activator of SHH signaling is a recombinant N-Terminal fragment of a full-length murine sonic hedgehog protein capable of binding to the SHH receptor for activating SHH. In some embodiments, the activator of SHH signaling is C25II SHH protein.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

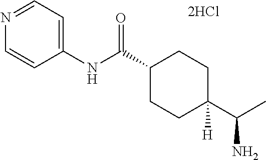 [SMILES: C[C@@H](N)[C@H]1CC[C@H](C(=O)Nc2ccncc2)CC1]

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of at or about 10 μM.

In some embodiments, cells are exposed to Y-27632 at a concentration of at or about 10 μM. In some embodiments, cells are exposed to Y-27632 at a concentration of at or about 10 μM on Day 0 of the first incubation as described below.

### Automated Non-Adherent “Suspension Culture” Differentiation Protocols in Cell Culture Bag

The provided methods include culturing PSCs (e.g., iPSCs) by incubation with certain molecules (e.g., small molecules) to induce their differentiation into floor plate midbrain progenitor cells, dopaminergic neuronal progenitor cells, including determined dopaminergic neuronal progenitor cells, committed dopaminergic neuronal progenitor cells and/or, dopaminergic neuronal cells. In particular, the provided embodiments include a first incubation of PSCs under non-adherent conditions to produce spheroids, in the presence of certain molecules (e.g., small molecules), in which the first incubation is performed while the cells are in a microwell cell culture bag, which can, in some aspects, improve the consistency of producing physiologically relevant cells for implantation, and also improve manufacturing by accelerating the timeline for differentiating the iPSCs into, e.g., dopaminergic neuronal progenitor cells, determined dopaminergic neuronal progenitor cells, committed dopaminergic neuronal progenitor cells, and/or dopaminergic neuronal cells, thereby advantageously reducing the amount of time and resources needed for the differentiation process.

In some embodiments, the methods include performing a first incubation involving culturing pluripotent stem cells in a cell culture bag under conditions to produce a cell spheroid, wherein (i) the pluripotent stem cells are exposed to at least one inhibitor of TGF-β/activin-Nodal signaling and at least one inhibitor of bone morphogenetic protein (BMP) signaling for at least one day (Day 0); and (ii) starting on the second day (Day 1) of the first incubation, exposing the pluripotent stem cells to at least one activator of Sonic Hedgehog (SHH) signaling and at least one inhibitor of glycogen synthase kinase 3β (GSK3β) signaling. In some embodiments, the pluripotent stem cells are exposed to a ROCK inhibitor such as Y-27632 at a concentration of at or about 10 μM on Day 0 of the first incubation. In some embodiments, the pluripotent stem cells were not exposed to a ROCKi prior to the start of the first incubation.

In some embodiments, the Day 0 incubation is done in the absence of x) an activator of Sonic Hedgehog (SHH) signaling, and y) an inhibitor of glycogen synthase kinase 3β (GSK3β) signaling, and the pluripotent stem cells are exposed to at least one activator of Sonic Hedgehog (SHH) signaling and at least one inhibitor of glycogen synthase kinase 3β (GSK3β) signaling starting on the second day of the first incubation (Day 1). This “Day 1” suspension culture method as described herein and in U.S. Provisional Application No. 63/472,789 entitled “METHODS FOR DIFFERENTIATING DOPAMINERGIC NEURONS FROM STEM CELLS,” filed Jun. 13, 2023. Exposing the cells to an inhibitor of TGF-β/activin-Nodal signaling (e.g., SB431542) and an inhibitor of bone morphogenetic protein (BMP) signaling (e.g., LDN193189) beginning on Day 0 when the cells are seeded, and then waiting until Day 1 to begin exposing the cells to at least one activator of Sonic Hedgehog (SHH) signaling (e.g., SHH protein and/or purmorphamine), and an inhibitor of glycogen synthase kinase 3β (GSK3β) signaling (e.g., CHIR99021) provides advantages over adherent methods and other non-adherent methods that may, for example, expose the cells to each of these agents beginning on Day 0. For instance, in some cases, cells exposed to these agents beginning on Day 0 or Day 1, as described above, advantageously exhibit one or more of: (i) increased expression of the dopaminergic lineage marker EN1 on or about Day 7 as compared to if the cells were exposed to each of these agents beginning on Day 0; (ii) reduced expression of the off-target non-dopaminergic lineage marker GBX2 on or about Day 7 as compared to if the cells were exposed to each of these agents beginning on Day 0; and (iii) increased expression of the dopaminergic lineage marker LMX1A on or about Day 10 as compared to if the cells were exposed to each of these agents beginning on Day 0. Accordingly, in some cases, exposing the cells to an inhibitor of TGF-β/activin-Nodal signaling (e.g., SB431542) and an inhibitor of BMP signaling (e.g., LDN193189) beginning on Day 0 when the cells are seeded, and then waiting until Day 1 to begin exposing the cells to at least one activator of SHH signaling (e.g., SHH protein and/or purmorphamine), and an inhibitor of GSK3β signaling (e.g., CHIR99021), advantageously results in increased specificity for the dopaminergic lineage as compared to if the cells were exposed to each of these agents beginning on Day 0.

Further aspects of the provided methods include harvesting cells at a time at which the cells are determined dopaminergic neuronal precursor cells or committed dopaminergic neuronal precursor cells that are cells that are able to differentiate into dopaminergic neurons but cannot differentiate into non-dopaminergic neurons. In some embodiments of the provided methods, such cells are cells that are differentiated in accord with the provided methods and are harvested between about Day 14 and about Day 17. This is earlier than other methods where differentiated determined dopaminergic neuronal precursor cells or committed dopaminergic progenitor cells are harvested at Day 20 or beyond, including Day 25.

In some embodiments, the activator of Sonic Hedgehog (SHH) signaling and/or the inhibitor of glycogen synthase kinase 3β (GSK3β) signaling are added later in the first incubation, at, for example, Day 2 through Day 7.

In some embodiments, the cell culture bag has a low or ultra-low attachment surface, such as to inhibit or reduce cell attachment. In some embodiments, culturing cells in a non-adherent culture vessel does not prevent all cells of the culture from attaching the surface of the culture vessel. In some embodiments, an ultra-low attachment surface may inhibit cell attachment for the period of time necessary to obtain confluent growth of the same cell type on an adherent surface. In some embodiments, the ultra-low attachment surface is coated or treated with a substance to prevent cell attachment, such as a hydrogel layer (e.g., a neutrally charged and/or hydrophilic hydrogel layer). In some embodiments, a non-adherent culture vessel is coated or treated with a surfactant prior to the first incubation. In some embodiments, the surfactant is pluronic acid.

In some embodiments, the non-adherent culture vessel allows for three-dimensional formation of cell aggregates. In some embodiments, iPSCs are cultured in a non-adherent culture vessel, such as a multiwell cell culture bag or a multiwell plate, to produce cell aggregates (e.g., spheroids). In some embodiments, iPSCs are cultured in a non-adherent culture vessel, such as a multi-well cell culture bag, to produce cell aggregates (e.g., spheroids) on about Day 7 of the method. In some embodiments, the cell aggregate (e.g., spheroid) expresses at least one of PAX6 and OTX2 on or by about Day 7 of the method.

In some embodiments, the non-adherent culture vessel is a closed-system cell culture vessel such as a microwell bag. Such microwell bags are described, for example, by Suenaga et al. (2022) “Microwell bag culture for large-scale production of homogenous islet-like clusters.” Scientific Reports 12: 5221. See also, e.g., US Patent Application 2023/0227787 and PCT Patent Application WO 2016/208526. Suitable microwell cell culture bags that are suitable for forming spheroids include, for example, the “WellBag™” ToyoSeikan Co., Ltd., which are available from, for example, Nacalai USA. Suitable cell culture bags typically have at least one port and a lower face that has a plurality of recesses (microwells). In some embodiments, each culture bag has approximately 18,000 microwells. Another example of a suitable non-adherent culture vessel is the Elplasia® round bottom plate (Corning).

The non-adherent suspension culture differentiation methods provided herein, in some embodiments, use a holder apparatus in conjunction with a microwell cell culture bags such as the Wellbag. The holder apparatus can include a pressure plate that can apply pressure to the microwell bag when the bag is placed in the holder. Suitable holders are described in, for example, US Patent Application 2023/0227787. As used herein, the phrase “applying a pressure” means that the liquid depth of the cell culture bag packed with cells and a predetermined culture medium is equalized.

In some embodiments, the number of PSCs introduced into the microwell cell culture bag on Day 0 of the method is between about 100 pluripotent stem cells per microwell and about 5,000 pluripotent stem cells per microwell. In some embodiments, the number of pluripotent stem cells per microwell is between about 250 and 3,000, and in some embodiments, the number of PSCs introduced into the microwell culture bag is between about 500 and 1,000 cells per microwell.

In some embodiments, the number of PSCs introduced into the microwell cell culture bag on Day 0 of the method is a number of cells sufficient to produce a cellular spheroid containing between about 500 cells and about 20,000 cells, or between about 1,000 cells and about 15,000 cells. In some embodiments, the number of PSCs introduced into the cell culture bag on Day 0 of the method is a number of cells sufficient to produce a cellular spheroid containing between about 1,000 cells and about 12,000 cells. In some embodiments, the number of PSCs introduced into the microwell cell culture bag on Day 0 of the method is a number of cells sufficient to produce a cellular spheroid containing between about 2,000 cells and about 3,000 cells. In some embodiments, the spheroids containing the desired number of cells are produced by the method on or by about Day 7.

In some embodiments of the method provided herein, the first incubation includes culturing pluripotent stem cells in a non-adherent cell culture bag under conditions to produce a cellular spheroid. In some embodiments, the first incubation is from about Day 0 through about Day 6. In some embodiments, the first incubation comprises culturing pluripotent stem cells in a culture media (“media”). In some embodiments, the media of the first incubation is a basal induction media for inducing differentiation of the PSCs into floor plate midbrain progenitor cells. In some embodiments, the first incubation comprises culturing pluripotent stem cells in the media from about Day 0 through about Day 6. In some embodiments, the first incubation comprises culturing pluripotent stem cells in the media to induce differentiation of the PSCs into floor plate midbrain progenitor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on Day 0 and Day 1. In some embodiments, the serum replacement is provided in the media at 2% (v/v) for at least a portion of the first incubation. In some embodiments, the serum replacement is provided in the media at 2% (v/v) from Day 2 through Day 6. In some embodiments, the serum replacement is provided in the media at 5% (v/v) on Day 0 and Day 1, and at 2% (v/v) from Day 2 through Day 6.

In some embodiments, the media is supplemented with a Rho-associated protein kinase (ROCK) inhibitor on one or more days when cells are passaged. In some embodiments, the media is supplemented with a ROCK inhibitor each day that cells are passaged. In some embodiments the media is supplemented with a ROCK inhibitor on Day 0. In some embodiments, the cells are not exposed to a ROCKi prior to exposing the pluripotent stem cells to the inhibitor of TGF-β/activin-Nodal signaling and the inhibitor of bone morphogenetic protein (BMP) signaling in the first incubation.

In some embodiments the media is supplemented with an inhibitor of TGF-β/activin-Nodal signaling. In some embodiments the media is supplemented with an inhibitor of TGF-β/activin-Nodal signaling up to about Day 5 (e.g., Day 4 or Day 5). In some embodiments the media is supplemented with an inhibitor of TGF-β/activin-Nodal signaling from about Day 0 through Day 4, each day inclusive.

In some embodiments, the media is partially or completely exchanged on one or more days of the first incubation, e.g., each of Days 1 through 6. In some embodiments, the media exchange comprises replacing at or about 50% of the media with fresh media, which can be the same as or different than the previous media. In some embodiments, the media exchange comprises replacing between about 25% and about 75% of the media. In some embodiments, the media on one or more days of the first incubation, e.g., each of Days 1 through 6, is added by a 50% media exchange. For instance, the media on one or more of Days 1 through 6 is added by removing about 50% of the previous media and replacing it with an equal volume of the new media prepared for that day. Accordingly, the concentration of certain small molecules included in the media to be added through a 50% media exchange is included in the new media at a double (2×) concentration of what the cells are intended to be exposed to on that day.

In some embodiments, all or nearly all of the media is exchanged by removing all or nearly all of the media from the cell culture bag without disturbing the growing spheroids that are present in the microwells. The media is then replaced with fresh media, which can be the same or different than the media being replaced. In some embodiments, the media exchange is accomplished by attaching a first port of the cell culture bag to a first port of a first pump; and attaching a media bag that contains fresh media to a second port of the first pump. A second port of the cell culture bag is attached to a first port of a second pump, and a waste bag is attached to a second port of the second pump. The media exchange is effected by using the first pump to inject the fresh media into the cell culture bag while the second pump removes media that was previously contained in the cell culture bag and transfers it to the waste bag. In some embodiments, the media being exchanged includes an inhibitor of TGF-β/activin-Nodal signaling and an inhibitor of bone morphogenetic protein (BMP) signaling, and the fresh media is Day 1 media that includes an activator of Sonic Hedgehog (SHH) signaling and an inhibitor of glycogen synthase kinase 3β (GSK3β). In some embodiments, the fresh media is the same as the media being replaced. In some embodiments, the media is exchanged on each of Days 1 through 6 of the first incubation.

In some embodiments, cells are exposed to the inhibitor of TGF-β/activin-Nodal signaling at a concentration of between about 1 μM and about 20 μM, between about 5 μM and about 15 μM, or between about 8 μM and about 12 μM. In some embodiments, cells are exposed to the inhibitor of TGF-β/activin-Nodal signaling at a concentration of at or about 10 μM. In some embodiments, cells are exposed to SB431542 at a concentration of about 10 μM. In some embodiments, cells are exposed to SB431542 at a concentration of about 10 μM until about Day 5. In some embodiments, cells are exposed to SB431542 at a concentration between about 8 μM and about 15 μM from about Day 0 through about Day 4, inclusive of each day. In some embodiments, cells are exposed to SB431542 at a concentration of at or about 10 μM from about Day 0 through about Day 4, inclusive of each day.

In some embodiments the media is supplemented with an inhibitor of BMP signaling. In some embodiments the media is supplemented with an inhibitor of BMP signaling up to about Day 7 (e.g., Day 6 or Day 7). In some embodiments the media is supplemented with an inhibitor of BMP signaling from about Day 0 through Day 6, each day inclusive. In some embodiments, cells are exposed to the inhibitor of BMP signaling at a concentration of between about 0.01 μM and about 5 μM, between about 0.05 μM and about 1 μM, between about 0.05 μM and about 0.2 μM, or between about 0.1 μM and about 0.5 μM, each inclusive. In some embodiments, cells are exposed to the inhibitor of BMP signaling at a concentration of at or about 0.1 μM.

In some embodiments, cells are exposed to LDN193189 at a concentration of about 0.1 μM. In some embodiments, cells are exposed to LDN193189 at a concentration of about 0.1 μM up to about Day 7 (e.g., Day 6 or Day 7). In some embodiments, cells are exposed to LDN193189 at a concentration of about 0.1 μM on Day 0 and are exposed to LDN193189 at a concentration of from about 0.08 μM to about 0.15 μM from about Day 0 through about Day 6, inclusive of each day. In some embodiments, cells are exposed to LDN193189 at a concentration of about 0.1 μM from about Day 0 through about Day 6, inclusive of each day.

In some embodiments the media is supplemented with an inhibitor of GSK3β signaling. In some embodiments the media is supplemented with an inhibitor of GSK3β signaling beginning on Day 1 and up to about Day 7 (e.g., Day 6 or Day 7). In some embodiments the media is supplemented with an inhibitor of GSK3β signaling from about Day 1 through Day 6, each day inclusive. In some embodiments, the cells are not exposed to an inhibitor of GSK3β signaling prior to Day 1 of the incubation process.

In some embodiments, cells are exposed to the inhibitor of GSK3β signaling at a concentration of between about 0.1 μM and about 10 μM, between about 0.5 μM and about 8 μM, between about 0.5 μM and about 2 μM, between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3β signaling at a concentration of between about 0.5 μM and about 2 μM. In some embodiments, cells are exposed to the inhibitor of GSK3β signaling at a concentration of between about 1 μM and about 4 μM. In some embodiments, cells are exposed to the inhibitor of GSK3β signaling at a concentration of between about 2 μM and about 3 μM. In some embodiments, cells are exposed to the inhibitor of GSK3β signaling at a concentration of about 1 μM on Day 1, and about 2 μM on each of Days 2 through 6. In some embodiments, cells are exposed to the inhibitor of GSK3β signaling on Day 1 at a concentration that is 50% of the concentration of the inhibitor of GSK3β signaling that the cells are exposed to on each of Days 2 through 6.

In some embodiments the media is supplemented with the at least one activator of SHH signaling from about Day 1 through Day 6, each day inclusive. In some embodiments, the cells are not exposed to an activator of sonic hedgehog (SHH) signaling prior to Day 1 of the differentiation protocol.

In some embodiments, cells are exposed to the at least one activator of SHH signaling at a concentration of between about 10 ng/mL and about 500 ng/mL, between about 20 ng/mL and 400 ng/mL, between about 30 ng/mL and about 300 ng/mL, between about 40 ng/mL and about 200 ng/mL, between about 50 ng/mL and 150 ng/mL, between about 50 ng/mL and about 100 ng/mL, or between about 75 ng/mL and 125 ng/mL, each inclusive. In some embodiments, cells are exposed to the at least one activator of SHH signaling at a concentration of between about 75 ng/mL and about 125 ng/mL, each inclusive. In some embodiments, cells are exposed to the at least one activator of SHH signaling at a concentration of between about 50 ng/mL and about 100 ng/mL, each inclusive. In some embodiments, cells are exposed to the at least one activator of SHH signaling at a concentration of at or about 100 ng/mL. In some embodiments, the cells are exposed to SHH protein at or about 100 ng/mL. In some embodiments, the cells are exposed to recombinant SHH protein at or about 100 ng/mL. In some embodiments, the cells are exposed to recombinant mouse SHH protein at or about 100 ng/mL. In some embodiments, the cells are exposed to C25II SHH protein at or about 100 ng/mL.

In some embodiments, cells are exposed to recombinant SHH protein at a concentration of at or about 100 ng/mL. In some embodiments, cells are exposed to recombinant SHH protein at a concentration of at or about 100 ng/mL beginning on Day 1 and up to about Day 7 (e.g., Day 6 or Day 7). In some embodiments, cells are exposed to recombinant SHH protein at a concentration of about 100 ng/mL from about Day 1 through about Day 6, inclusive of each day.

In some embodiments, cells are exposed to the at least one activator of SHH signaling at a concentration of between about 1 μM and about 20 μM, between about 5 μM and about 15 μM, or between about 8 μM and about 12 μM. In some embodiments, cells are exposed to the at least one activator of SHH signaling at a concentration of between about 1 μM and about 20 μM. In some embodiments, cells are exposed to the at least one activator of SHH signaling at a concentration of between about 5 μM and about 15 μM. In some embodiments, cells are exposed to the at least one activator of SHH signaling at a concentration of between about 8 μM and about 12 μM. In some embodiments, cells are exposed to the at least one activator of SHH signaling at a concentration of about 10 μM.

In some embodiments, cells are exposed to purmorphamine at a concentration of about 2 μM. In some embodiments, cells are exposed to purmorphamine at a concentration of about 2 μM beginning on Day 1 and up to Day 7 (e.g., Day 6 or Day 7). In some embodiments, cells are exposed to purmorphamine at a concentration of about 2 μM from about Day 1 through about Day 6, inclusive of each day.

In some embodiments, cells are exposed to purmorphamine at a concentration of between about 0.1 μM and about 20 μM, between about 0.5 μM and about 10 μM, between about 1 μM and about 5 μM, between about 1 μM and about 3 μM, or between about 1.5 μM and about 2.5 μM. In some embodiments, cells are exposed to purmorphamine at a concentration of about 2 μM.

In some embodiments, the at least one activator of SHH signaling is SHH protein and purmorphamine. In some embodiments, cells are exposed to SHH protein and purmorphamine beginning on Day 1 and up to about Day 7 (e.g., Day 6 or Day 7). In some embodiments, cells are exposed to SHH protein and purmorphamine from about Day 1 through about Day 6, inclusive of each day. In some embodiments, cells are exposed to about 100 ng/mL SHH protein and about 2 μM purmorphamine beginning on Day 1 and up to about Day 7 (e.g., Day 6 or Day 7). In some embodiments, cells are exposed to about 100 ng/mL SHH protein and about 2 μM purmorphamine from about Day 1 through about Day 6, inclusive of each day.

In some embodiments, the first incubation comprises culturing pluripotent stem cells in a “basal induction media.” In some embodiments, the first incubation comprises culturing pluripotent stem cells in the basal induction media from about Day 0 through about Day 6. In some embodiments, the first incubation comprises culturing pluripotent stem cells in the basal induction media to induce differentiation of the PSCs into floor plate midbrain progenitor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the small molecules as described above.

In some aspects, methods involving the provided non-adherent culture methods described herein provide for advantages over adherent culture methods and other non-adherent culture methods with regards to manufacturability, including reduced cost and reduced time involved, by accelerating the differentiation timeline as compared to adherent culture methods. For instance, PSC-derived differentiated dopaminergic neurons cultured in accordance with the non-adherent culture methods described herein may exhibit a gene expression profile on, e.g., Day 16, that is comparable to the gene expression profile of, e.g., Day 20 or Day 21, of PSC-derived differentiated dopaminergic neurons cultured using an adherent culture method. This can advantageously reduce the amount of resources, including media, inhibitors, and other supplements, that are required, and can also advantageously reduce the amount of time required to produce PSC-derived differentiated dopaminergic neurons, e.g., by allowing for harvesting or collection between approximately Days 14-17, e.g., on or about Day 14, 15, 16, or 17.

Further, particular benefits are associated with methods of differentiating cells that include a non-adherent culture of only about 7 days. The non-adherent culture of about 7 days described herein produces spheroids. If non-adherent culture is allowed to proceed beyond 7 days, the increasing size of the spheroids may limit mass transport and result in reagent (e.g., morphogen) gradients across the diameter of the spheroids. Substantial reagent (e.g., morphogen) gradients across the diameter of the spheroids is undesirable, as different cells of the spheroids would be exposed to different concentration of a reagent (e.g., a morphogen), thereby leading to variable cell response and differentiation. Thus, the methods described herein including non-adherent culture of only about or up to 7 days are advantageous because variability in the concentration of reagents such as morphogen(s) to which the cells of the spheroids are exposed is minimized.

In some aspects, limiting the non-adherent culture component of the methods described herein to about 7 days also helps ensure the consistent differentiation of cultured cells. This is because long-term (e.g., greater than about 7 days) differentiation of PSCs in non-adherent (e.g., suspension) culture may allow PSCs to establish a microenvironment that allows for self-renewal in a pluripotent state. Thus, the methods described herein ensure the consistent and effective differentiation of the cultured cells by reducing or eliminating the opportunity for PSCs to persist in culture.

Transfer and/or Dissociation of Spheroids

In some embodiments, cell aggregates (e.g., spheroids) that are produced following the first incubation of culturing pluripotent stem cells in a microwell cell culture bag are transferred or dissociated, prior to carrying out a second incubation of the cells under adherent culture. The spheroids can be recovered from the culture bag by, for example, inverting the bag so that the face of the bag that includes the multiwells is on top, introducing a volume of air into the bag and then aspirating the spheroid-containing media from the bag using a syringe.

In some embodiments, the first incubation is carried out to produce a cell aggregate (e.g., a spheroid) that expresses at least one of PAX6 and OTX2. In some embodiments, the first incubation produces a cell aggregate (e.g., a spheroid) that expresses PAX6 and OTX2. In some embodiments, the first incubation produces a cell aggregate (e.g., a spheroid) on or by about Day 7 of the methods provided herein. In some embodiments, the first incubation produces a cell aggregate (e.g., a spheroid) that expresses at least one of PAX6 and OTX2 on or by about Day 7 of the methods provided herein. In some embodiments, the first incubation produces a cell aggregate (e.g., a spheroid) that expresses PAX6 and OTX2 on or by about Day 7 of the methods provided herein.

In some embodiments, the cell aggregate (e.g., spheroid) produced by the first incubation is dissociated prior to the second incubation of the cells under adherent conditions. In some embodiments, the cell aggregate (e.g., spheroid) produced by the first incubation is dissociated to produce a cell suspension. In some embodiments, the cell suspension produced by the dissociation is a single cell suspension. In some embodiments, the dissociation is carried out at a time when the spheroid cells express at least one of PAX6 and OTX2. In some embodiments, the dissociation is carried out at a time when the spheroid cells express PAX6 and OTX2. In some embodiments, the dissociation is carried out on about Day 7. In some embodiments, the cell aggregate (e.g., spheroid) is dissociated by enzymatic dissociation. In some embodiments, the enzyme is selected from among the group consisting of: Accutase®, dispase, collagenase, and combinations thereof. In some embodiments, the enzyme comprises Accutase®. In some embodiments, the enzyme is Accutase®. In some embodiments, the enzyme is dispase. In some embodiments, the enzyme is collagenase. In some embodiments, the enzyme is dispase and collagenase.

### Adherent Cell Culture of Spheroid-Derived Cells

In some embodiments, the cell aggregate or cell suspension produced by the non-adherent suspension culture protocol is transferred to a second culture vessel for a second incubation under adherent conditions. In some embodiments, the cell aggregate (e.g., spheroid) or cell suspension produced therefrom is transferred to a substrate-coated culture vessel following dissociation of the cell aggregate (e.g., spheroid). In some embodiments, the transferring is carried out immediately after the dissociating. In some embodiments, the transferring is carried out on about Day 7.

In some embodiments, the cell aggregate (e.g., spheroid) is not dissociated prior to a second incubation. In some embodiments, a cell aggregate (e.g., spheroid) is transferred in its entirety to a second culture vessel for a second incubation in which the cells adhere to the second culture vessel. In some embodiments, the transferring is carried out at a time when the spheroid cells express at least one of PAX6 and OTX2. In some embodiments, the transferring is carried out at a time when the spheroid cells express PAX6 and OTX2. In some embodiments, the transferring is carried out on about Day 7.

In some embodiments, the cell aggregate or cell suspension is transferred to a second culture vessel that is treated so that the cells adhere to the culture vessel. In some embodiments, the culture vessel is a plate, a dish, a flask, or a bioreactor. In some embodiments, the culture vessel is substrate-coated. In some embodiments, the substrate is a basement membrane protein. In some embodiments, the substrate is selected from laminin or a fragment thereof, collagen, entactin, heparin sulfate proteoglycans, and combinations thereof. In some embodiments, the substrate is laminin. In some embodiments, the substrate is recombinant. In some embodiments, the substrate is recombinant laminin or a fragment thereof. In some embodiments, the substrate is xeno-free. In some embodiments, the substrate is xeno-free laminin or a fragment thereof.

In some embodiments, the laminin or fragment thereof comprises an alpha chain, a beta chain, and a gamma chain. In some embodiments, the alpha chain is LAMA1, LAMA2, LAMA3, LAMA4, LAMA5, or a combination thereof. In some embodiments, the beta chain is LAMB1, LAMB2, LAMB3, LAMB4, or a combination thereof. In some embodiments, the gamma chain is LAMC1, LAMC2, LAMC3, or a combination thereof. In some embodiments, the laminin or a fragment thereof comprises any alpha, beta, and/or gamma chains as described in Aumailley, (2013) Cell Adh Migra 7(1):48-55 (see e.g. Table 1).

In some embodiments, the laminin or a fragment thereof is selected from the group consisting of: laminin 111, laminin 121, laminin 211, laminin 213, laminin 221, laminin 3A32, laminin 3B32, laminin 3A11, laminin 3A21, laminin 411, laminin 421, laminin 423, laminin 511, laminin 521, laminin 522, laminin 523, or a fragment of any of the foregoing. In some embodiments, the laminin is selected from laminin 521, laminin 111, laminin 511, and laminin 511-E8. In some embodiments, the laminin or a fragment thereof comprises an E8 fragment of LAMA5, an E8 fragment of LAMB1, and an E8 fragment of LAMC1. In some embodiments, the laminin or a fragment thereof is laminin 511-E8 fragment. See Miyazaki et al., (2012) Nat Commun 3:1236. In some embodiments, the substrate-coated culture vessel is exposed to poly-L-ornithine, optionally prior to being used for culturing cells.

In some embodiments, the substrate-coated culture vessel is a plate, a dish, a flask, or a bioreactor. In some embodiments, the substrate-coated culture vessel is a 6-well, 12-well, or 24-well plate. In some embodiments, the substrate-coated culture vessel is a 6-well plate. In some embodiments, the substrate-coated culture vessel is a 12-well plate. In some embodiments, the substrate-coated culture vessel is a 24-well plate.

In some embodiments, the second incubation involves culturing cells of the spheroid in a second culture vessel that is coated with a substrate that promotes adhesion of the cells to the culture vessel. Beginning when the cells are placed in the second culture vessel, typically on Day 7, the cells are exposed to (i) an inhibitor of BMP signaling and (ii) an inhibitor of GSK3β signaling; and beginning on or about Day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the second culture vessel allows for a monolayer cell culture. In some embodiments, cells derived from the cell aggregate (e.g., spheroid) produced by the first incubation described in B.3 above are cultured in a monolayer culture on the second culture vessel. In some embodiments, cells derived from the cell aggregate (e.g., spheroid) produced by the first incubation are cultured to produce a monolayer culture of cells positive for one or more of LMX1A, FOXA2, EN1, CORIN, and combinations thereof. In some embodiments, cells derived from the cell aggregate (e.g., spheroid) produced by the first incubation are cultured to produce a monolayer culture of cells, wherein at least some of the cells are TH+. In some embodiments, at least some cells are TH+ by or on about Day 16. In some embodiments, cells derived from the cell aggregate (e.g., spheroid) produced by the first incubation are cultured to produce a monolayer culture of cells, wherein at least some of the cells are TH+FOXA2+. In some embodiments, at least some cells are TH+FOXA2+ by or on about Day 16.

In the methods provided herein, the second incubation involves culturing cells of the spheroid in a second culture vessel under conditions to induce neural differentiation of the cells. In some embodiments, the cells of the spheroid are plated on the second culture vessel on about Day 7.

In some embodiments, the number of cells plated on the second culture vessel is between about 0.1×10⁶ cells/cm² and about 2×10⁶ cells/cm², between about 0.1×10⁶ cells/cm² and about 1.5×10⁶ cells/cm², between about 0.1×10⁶ cells/cm² and about 1×10⁶ cells/cm², between about 0.1×10⁶ cells/cm² and about 0.8×10⁶ cells/cm², between about 0.2×10⁶ cells/cm² and about 2×10⁶ cells/cm², between about 0.2×10⁶ cells/cm² and about 1.5×10⁶ cells/cm², between about 0.2×10⁶ cells/cm² and about 1×10⁶ cells/cm², between about 0.2×10⁶ cells/cm² and about 0.8×10⁶ cells/cm², between about 0.4×10⁶ cells/cm² and about 2×10⁶ cells/cm², between about 0.4×10⁶ cells/cm² and about 1.5×10⁶ cells/cm², between about 0.4×10⁶ cells/cm² and about 1×10⁶ cells/cm², between about 0.4×10⁶ cells/cm² and about 0.8×10⁶ cells/cm², between about 0.6×10⁶ cells/cm² and about 2×10⁶ cells/cm², between about 0.6×10⁶ cells/cm² and about 1.5×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², or between about 0.8×10⁶ cells/cm² and about 1×10⁶ cells/cm². In some embodiments, the number of cells plated on the second culture vessel is between about 0.6×10⁶ cells/cm² and about 1.0×10⁶ cells/cm². In some embodiments, the number of cells plated on the second culture vessel is at or about 0.8×10⁶ cells/cm².

In some embodiments, the second incubation involves culturing cells derived from the cell aggregate (e.g., spheroid) in a culture media (“media”).

In some embodiments, the second incubation involves culturing the cells derived from the cell aggregate in the media from about Day 7 until harvest or collection. In some embodiments, the media is at least partially exchanged on each day beginning on Day 8 until harvest or collection. In the media exchange, new media replaces the media added on the prior day, e.g., there is a complete or near complete replacement or exchange of the media on each of these days. In some embodiments, cells are cultured in the media to produce determined dopaminergic neuronal progenitor cells, committed dopaminergic neuronal progenitor cells or dopaminergic neuronal cells.

In some embodiments, the media is also supplemented with a serum replacement containing minimal non-human-derived components (e.g., KnockOut™ serum replacement). In some embodiments, the media is supplemented with the serum replacement from about Day 7 through about Day 10. In some embodiments, the media is supplemented with about 2% (v/v) of the serum replacement. In some embodiments, the media is supplemented with about 2% (v/v) of the serum replacement from about Day 7 through about Day 10.

In some embodiments, the media is further supplemented with small molecules. In some embodiments, the small molecules are selected from among the group consisting of: a Rho-associated protein kinase (ROCK) inhibitor, an inhibitor of bone morphogenetic protein (BMP) signaling, an inhibitor of glycogen synthase kinase 33 (GSK3β) signaling, and combinations thereof.

In some embodiments the media is supplemented with a Rho-associated protein kinase (ROCK) inhibitor on one or more days when cells are passaged. In some embodiments, the cells are passaged on or about Day 7. In some embodiments the media is supplemented with a ROCK inhibitor each day that cells are passaged, e.g., on Day 7. In some embodiments the media is supplemented with a ROCK inhibitor on Day 7.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of at or about 10 μM. In some embodiments, cells are exposed to Y-27632 at a concentration of about 10 μM. In some embodiments, cells are exposed to Y-27632 at a concentration of at or about 10 μM on Day 7.

In some embodiments the media is supplemented with an inhibitor of BMP signaling. In some embodiments the media is supplemented with an inhibitor of BMP signaling from about Day 7 up to about Day 11 (e.g., Day 10 or Day 11). In some embodiments the media is supplemented with an inhibitor of BMP signaling from about Day 7 through Day 10, each day inclusive.

In some embodiments, cells are exposed to the inhibitor of BMP signaling at a concentration of between about 0.01 μM and about 5 μM, between about 0.05 μM and about 1 μM, between about 0.05 μM and about 0.2 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.05 μM and about 0.2 μM. In some embodiments, cells are exposed to the inhibitor of BMP signaling at a concentration of about 0.1 μM. In some embodiments, cells are exposed to LDN193189 at a concentration of about 0.1 μM. In some embodiments, cells are exposed to LDN193189 at a concentration of about 0.1 μM from about Day 7 up to about Day 11 (e.g., Day 10 or Day 11). In some embodiments, cells are exposed to LDN193189 at a concentration of about 0.1 μM from about Day 7 through about Day 10, inclusive of each day.

In some embodiments the media is supplemented with an inhibitor of GSK3β signaling. In some embodiments the media is supplemented with an inhibitor of GSK3β signaling from about Day 7 up to about Day 13 (e.g., Day 12 or Day 13). In some embodiments the media is supplemented with an inhibitor of GSK3β signaling from about Day 7 through Day 12, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, between about 1.5 μM and about 3 μM, or between about 1.5 μM and about 2.5 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3β signaling at a concentration of between about 1 μM and about 4 μM. In some embodiments, cells are exposed to the inhibitor of GSK3β signaling at a concentration of between about 1.5 μM and about 3 μM. In some embodiments, cells are exposed to the inhibitor of GSK3β signaling at a concentration of about 2 μM. In some embodiments, cells are exposed to CHIR99021 at a concentration of about 2.0 μM. In some embodiments, cells are exposed to CHIR99021 at a concentration of about 2.0 μM from about Day 7 up to about Day 13 (e.g., Day 12 or Day 13). In some embodiments, cells are exposed to CHIR99021 at a concentration of about 2.0 μM from about Day 7 through about Day 12, inclusive of each day.

In some embodiments the media is supplemented with brain-derived neurotrophic factor (BDNF). In some embodiments the media is supplemented with BDNF beginning on about Day 11. In some embodiments the media is supplemented with BDNF from about Day 11 until harvest or collection. In some embodiments the media is supplemented with BDNF from about Day 11 through about Day 14, 15, 16, or 17. In some embodiments the media is supplemented with BDNF from about Day 11 through Day 14. In some embodiments the media is supplemented with BDNF from about Day 11 through Day 15. In some embodiments the media is supplemented with BDNF from about Day 11 through Day 16. In some embodiments the media is supplemented with BDNF from about Day 11 through Day 17.

In some embodiments, cells are exposed to BDNF at a concentration of between about 1 ng/mL and 100 ng/mL, between about 5 ng/mL and about 50 ng/mL, between about 10 ng/mL and about 30 ng/mL. In some embodiments, cells are exposed to BDNF at a concentration of between about 10 ng/mL and about 30 ng/mL. In some embodiments, cells are exposed to BDNF at a concentration of about 20 ng/mL.

In some embodiments, the media is supplemented with about 20 ng/mL BDNF beginning on about Day 11. In some embodiments the media is supplemented with 20 ng/mL BDNF from about Day 11 until harvest or collection. In some embodiments the media is supplemented with about 20 ng/mL BDNF from about Day 11 through about Day 14, 15, 16, or 17. In some embodiments the media is supplemented with about 20 ng/mL BDNF from about Day 11 through Day 14. In some embodiments the media is supplemented with about 20 ng/mL BDNF from about Day 11 through Day 15. In some embodiments the media is supplemented with about 20 ng/mL BDNF from about Day 11 through Day 16. In some embodiments the media is supplemented with about 20 ng/mL BDNF from about Day 11 through Day 17.

In some embodiments the media is supplemented with glial cell-derived neurotrophic factor (GDNF). In some embodiments the media is supplemented with GDNF beginning on about Day 11. In some embodiments the media is supplemented with GDNF from about Day 11 until harvest or collection. In some embodiments the media is supplemented with GDNF from about Day 11 through about Day 14, 15, 16, or 17. In some embodiments the media is supplemented with GDNF from about Day 11 through Day 14. In some embodiments the media is supplemented with GDNF from about Day 11 through Day 15. In some embodiments the media is supplemented with GDNF from about Day 11 through Day 16. In some embodiments the media is supplemented with GDNF from about Day 11 through Day 17.

In some embodiments, cells are exposed to GDNF at a concentration of between about 1 ng/mL and 100 ng/mL, between about 5 ng/mL and about 50 ng/mL, between about 10 ng/mL and about 30 ng/mL. In some embodiments, cells are exposed to GDNF at a concentration of between about 10 ng/mL and about 30 ng/mL. In some embodiments, cells are exposed to GDNF at a concentration of about 20 ng/mL.

In some embodiments, the media is supplemented with about 20 ng/mL GDNF beginning on about Day 11. In some embodiments the media is supplemented with 20 ng/mL GDNF from about Day 11 until harvest or collection. In some embodiments the media is supplemented with about 20 ng/mL GDNF from about Day 11 through about Day 14, 15, 16, or 17. In some embodiments the media is supplemented with about 20 ng/mL GDNF from about Day 11 through Day 14. In some embodiments the media is supplemented with about 20 ng/mL GDNF from about Day 11 through Day 15. In some embodiments the media is supplemented with about 20 ng/mL GDNF from about Day 11 through Day 16. In some embodiments the media is supplemented with about 20 ng/mL GDNF from about Day 11 through Day 17.

In some embodiments the media is supplemented with ascorbic acid. In some embodiments the media is supplemented with ascorbic acid beginning on about Day 11. In some embodiments the media is supplemented with ascorbic acid from about Day 11 until harvest or collection. In some embodiments the media is supplemented with ascorbic acid from about Day 11 through about Day 14, 15, 16, or 17. In some embodiments the media is supplemented with ascorbic acid from about Day 11 through Day 14. In some embodiments the media is supplemented with ascorbic acid from about Day 11 through Day 15. In some embodiments the media is supplemented with ascorbic acid from about Day 11 through Day 16. In some embodiments the media is supplemented with ascorbic acid from about Day 11 through Day 17.

In some embodiments, cells are exposed to ascorbic acid at a concentration of between about 0.05 mM and 5 mM, between about 0.1 mM and about 1 mM, between about 0.2 mM and about 0.5 mM, each inclusive. In some embodiments, cells are exposed to ascorbic acid at a concentration of between about 0.05 mM and about 5 mM, each inclusive. In some embodiments, cells are exposed to ascorbic acid at a concentration of between about 0.1 mM and about 1 mM, each inclusive. In some embodiments, cells are exposed to ascorbic acid at a concentration of about 0.2 mM.

In some embodiments, the media is supplemented with about 0.2 mM ascorbic acid beginning on about Day 11. In some embodiments the media is supplemented with 0.2 mM ascorbic acid from about Day 11 until harvest or collection. In some embodiments the media is supplemented with about 0.2 mM ascorbic acid from about Day 11 through about Day 14, 15, 16, or 17. In some embodiments the media is supplemented with about 0.2 mM ascorbic acid from about Day 11 through Day 14. In some embodiments the media is supplemented with about 0.2 mM ascorbic acid from about Day 11 through Day 15. In some embodiments the media is supplemented with about 0.2 mM ascorbic acid from about Day 11 through Day 16. In some embodiments the media is supplemented with about 0.2 mM ascorbic acid from about Day 11 through Day 17.

In some embodiments the media is supplemented with dibutyryl cyclic AMP (dbcAMP). In some embodiments the media is supplemented with dbcAMP beginning on about Day 11. In some embodiments the media is supplemented with dbcAMP from about Day 11 until harvest or collection. In some embodiments the media is supplemented with dbcAMP from about Day 11 through about Day 14, 15, 16, or 17. In some embodiments the media is supplemented with dbcAMP from about Day 11 through Day 14. In some embodiments the media is supplemented with dbcAMP from about Day 11 through Day 15. In some embodiments the media is supplemented with dbcAMP from about Day 11 through Day 16. In some embodiments the media is supplemented with dbcAMP from about Day 11 through Day 17.

In some embodiments, cells are exposed to dbcAMP at a concentration of between about 0.05 mM and 5 mM, between about 0.1 mM and about 3 mM, between about 0.2 mM and about 1 mM, each inclusive. In some embodiments, cells are exposed to dbcAMP at a concentration of between about 0.1 mM and about 3 mM, each inclusive. In some embodiments, cells are exposed to dbcAMP at a concentration of between about 0.2 mM and about 1 mM, each inclusive. In some embodiments, cells are exposed to dbcAMP at a concentration of about 0.5 mM.

In some embodiments, the media is supplemented with about 0.5 mM dbcAMP beginning on about Day 11. In some embodiments the media is supplemented with 0.5 mM dbcAMP from about Day 11 until harvest or collection. In some embodiments the media is supplemented with about 0.5 mM dbcAMP from about Day 11 through about Day 14, 15, 16, or 17. In some embodiments the media is supplemented with about 0.5 mM dbcAMP from about Day 11 through Day 14. In some embodiments the media is supplemented with about 0.5 mM dbcAMP from about Day 11 through Day 15. In some embodiments the media is supplemented with about 0.5 mM dbcAMP from about Day 11 through Day 16. In some embodiments the media is supplemented with about 0.5 mM dbcAMP from about Day 11 through Day 17.

In some embodiments the media is supplemented with transforming growth factor beta 3 (TGFβ3). In some embodiments the media is supplemented with TGFβ3 beginning on about Day 11. In some embodiments the media is supplemented with TGFβ3 from about Day 11 until harvest or collection. In some embodiments the media is supplemented with TGFβ3 from about Day 11 through about Day 14, 15, 16, or 17. In some embodiments the media is supplemented with TGFβ3 from about Day 11 through Day 14. In some embodiments the media is supplemented with TGFβ3 from about Day 11 through Day 15. In some embodiments the media is supplemented with TGFβ3 from about Day 11 through Day 16. In some embodiments the media is supplemented with TGFβ3 from about Day 11 through Day 17.

In some embodiments, cells are exposed to TGFβ3 at a concentration of between about 0.1 ng/mL and 10 ng/mL, between about 0.5 ng/mL and about 5 ng/mL, or between about 1.0 ng/mL and about 2.0 ng/mL. In some embodiments, cells are exposed to TGFβ3 at a concentration of between about 1.0 ng/mL and about 2.0 ng/mL, each inclusive. In some embodiments, cells are exposed to TGFβ3 at a concentration of about 1 ng/mL.

In some embodiments, the media is supplemented with about 1 ng/mL TGFβ3 beginning on about Day 11. In some embodiments the media is supplemented with 1 ng/mL TGFβ3 from about Day 11 until harvest or collection. In some embodiments the media is supplemented with about 1 ng/mL TGFβ3 from about Day 11 through about Day 14, 15, 16, or 17. In some embodiments the media is supplemented with about 1 ng/mL TGFβ3 from about Day 11 through Day 14. In some embodiments the media is supplemented with about 1 ng/mL TGFβ3 from about Day 11 through Day 15. In some embodiments the media is supplemented with about 1 ng/mL TGFβ3 from about Day 11 through Day 16. In some embodiments the media is supplemented with about 1 ng/mL TGFβ3 from about Day 11 through Day 17.

In some embodiments the media is supplemented with an inhibitor of Notch signaling. In some embodiments the media is supplemented with an inhibitor of Notch signaling beginning on about Day 11. In some embodiments the media is supplemented with an inhibitor of Notch signaling from about Day 11 until harvest or collection. In some embodiments the media is supplemented with an inhibitor of Notch signaling from about Day 11 through about Day 14, 15, 16, or 17. In some embodiments the media is supplemented with an inhibitor of Notch signaling from about Day 11 through Day 14. In some embodiments the media is supplemented with an inhibitor of Notch signaling from about Day 11 through Day 15. In some embodiments the media is supplemented with an inhibitor of Notch signaling from about Day 11 through Day 16. In some embodiments the media is supplemented with an inhibitor of Notch signaling from about Day 11 through Day 17.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

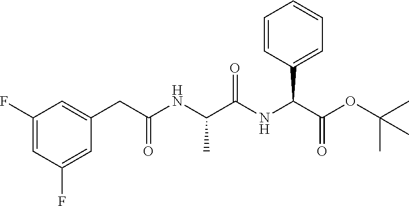 [SMILES: C[C@H](NC(=O)Cc1cc(F)cc(F)c1)C(=O)N[C@H](C(=O)OC(C)(C)C)c1ccccc1]

In some embodiments, cells are exposed to DAPT at a concentration of between about 1 μM and about 20 μM, between about 5 μM and about 15 μM, or between about 8 μM and about 12 μM. In some embodiments, cells are exposed to DAPT at a concentration of between about 1 μM and about 20 μM. In some embodiments, cells are exposed to DAPT at a concentration of between about 5 μM and about 15 μM. In some embodiments, cells are exposed to DAPT at a concentration of between about 8 μM and about 12 μM. In some embodiments, cells are exposed to DAPT at a concentration of about 10 μM.

In some embodiments, the media is supplemented with about 10 μM DAPT beginning on about Day 11. In some embodiments the media is supplemented with 10 μM DAPT from about Day 11 until harvest or collection. In some embodiments the media is supplemented with about 10 μM DAPT from about Day 11 through about Day 14, 15, 16, or 17. In some embodiments the media is supplemented with about 10 μM DAPT from about Day 11 through Day 14. In some embodiments the media is supplemented with about 10 μM DAPT from about Day 11 through Day 15. In some embodiments the media is supplemented with about 10 μM DAPT from about Day 11 through Day 16. In some embodiments the media is supplemented with about 10 μM DAPT from about Day 11 through Day 17.

In some embodiments, beginning on about Day 11, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about Day 11 until harvest or collection, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about Day 11 until about Day 14, 15, 16, or 17, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about Day 11 until Day 14, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about Day 11 until Day 15, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about Day 11 until Day 16, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about Day 11 until Day 17, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT.

In some embodiments, a serum replacement is provided in the media from about Day 7 through about Day 10. In some embodiments, the serum replacement is provided at 2% (v/v) in the media on Day 7 through Day 10.

In some embodiments, from about Day 7 to harvest or collection, e.g., about Day 14, 15, 16, or 17, the media is replaced daily. In some embodiments, from about Day 7 to harvest or collection, e.g., about Day 14, 15, 16, or 17, at least 90%, 91%, 92%, 93%, 94%, 95%, 96%, 97%, 98%, or 99% of the media is replaced daily. In some embodiments, from about Day 7 to harvest or collection, e.g., about Day 14, 15, 16, or 17, at least 95%, 96%, 97%, 98%, or 99% of the media is replaced daily. In some embodiments, from about Day 7 to harvest or collection, e.g., about Day 14, 15, 16, or 17, at least 98% or 99% of the media is replaced daily.

In some embodiments, the second incubation involves culturing cells derived from the cell aggregate (e.g., spheroid) in a “basal induction media.” In some embodiments, the second incubation involves culturing cells derived from the cell aggregate (e.g., spheroid) in a “maturation media.” In some embodiments, the second incubation involves culturing cells derived from the cell aggregate (e.g., spheroid) in the basal induction media, and then in the maturation media.

In some embodiments, the second incubation involves culturing the cells in the basal induction media from about Day 7 through about Day 10. In some embodiments, the second incubation involves culturing the cells in the maturation media beginning on about Day 11. In some embodiments, the second incubation involves culturing the cells in the basal induction media from about Day 7 through about Day 10, and then in the maturation media beginning on about Day 11. In some embodiments, the second incubation involves culturing the cells in the basal induction media from about Day 7 through about Day 10, and then in the maturation media beginning on about Day 11 until collection or harvest. In some embodiments, cells are cultured in the maturation media to produce determined dopaminergic neuronal progenitor cells, committed dopaminergic neuronal progenitor cells, and/or dopaminergic neuronal cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the molecules described in Section B.

In some embodiments, the maturation media is formulated to contain Neurobasal™ media, supplemented with N-2 and B27 supplements, non-essential amino acids (NEAA), and GlutaMAX™. In some embodiments, the maturation media is further supplemented with any of the molecules described in Section B.

In some embodiments, the cells are cultured in the basal induction media from about Day 7 up to about Day 11 (e.g., Day 10 or Day 11). In some embodiments, the cells are cultured in the basal induction media from about Day 7 through Day 10, each day inclusive. In some embodiments, the cells are cultured in the maturation media beginning on about Day 11. In some embodiments, the cells are cultured in the basal induction media from about Day 7 through about Day 10, and then the cells are cultured in the maturation media beginning on about Day 11. In some embodiments, the cells are cultured in the maturation media from about Day 11 until harvest or collection of the cells.

In some embodiments, the second incubation is from about Day 7 until harvesting of the cells. In some embodiments, the method further includes harvesting the differentiated cells. In some embodiments, the differentiated cells are harvested between about Day 14 and about Day 17. In some embodiments, the differentiated cells are harvested on or about Day 14, Day 15, Day 16, or Day 17. In some embodiments, the differentiated cells are harvested on or about Day 14. In some embodiments, the differentiated cells are harvested on or about Day 15. In some embodiments, the differentiated cells are harvested on or about Day 16. In some embodiments, the differentiated cells are harvested on or about Day 17. In some embodiments, the second incubation is from about Day 7 until about Day 14. In some embodiments, the second incubation is from about Day 7 until about Day 15. In some embodiments, the second incubation is from about Day 7 until about Day 16. In some embodiments, the second incubation is from about Day 7 until about Day 17.

In some embodiments, cells produced using the methods described herein, or therapeutic compositions containing such cells, may exhibit an improved ability to engraft and/or innervate other cells compared to cells harvested at later times of the differentiation method (e.g., Day 25). In some embodiments, cells harvested between days 14 and 17 may also exhibit improved efficacy in vivo, due to their state of differentiation and neuronal commitment. For example, cells harvested on Day 16 may demonstrate improved engraftment and/or innervation, improved efficacy, or both, as compared to cells harvested on Day 25. Accordingly, cells differentiated using the methods described herein may demonstrate improved engraftment and/or innervation, improved efficacy, or both, in addition to its improved manufacturability through reduced time and resources, including cost, involved.

### Adherent Culture Using Automated Cell Culture Systems

In some embodiments, the adherent culture portion of the differentiation protocol described herein is performed using an automated cell culture system. Suitable automated cell culturing systems are described in, for example, US Patent Application No. 2021/0238532. The automated cell culture systems are useful not only for the second incubation of the non-adherent suspension culture method described above in Section B.3, but also for a dopaminergic neuronal progenitor cell differentiation protocol in which both the first incubation and the second incubation are performed with the cells adhered to a substrate.

In some embodiments, the invention provides methods of differentiating pluripotent stem cells into dopaminergic neuronal progenitor cells, wherein the methods involve: (a) performing a first incubation that includes culturing pluripotent stem cells in a first culture vessel, wherein the first incubation includes: (i) exposing the pluripotent stem cells to an inhibitor of TGF-β/activin-Nodal signaling and an inhibitor of bone morphogenetic protein (BMP) signaling for at least one day (Day 0); and (ii) exposing the pluripotent stem cells to at least one activator of Sonic Hedgehog (SHH) signaling and an inhibitor of glycogen synthase kinase 3β (GSK3β) signaling; and (b) performing a second incubation comprising adherently culturing the cells in a second culture vessel under conditions to further differentiate the cells into dopaminergic neuronal progenitor cells, wherein at least one of the first incubation and the second incubation is performed using an automated cell culture system. In some embodiments, both the first and second incubations are performed using an automated cell culture system.

In some embodiments, the second incubation is performed using an automated cell culture system. The automated cell culture system includes at least a first cell culture container configured to receive a sample therein, and a first lid coupled to the first cell culture container, wherein the first lid comprises a first liquid exchange port and a first gas exchange port. The system also includes at least a first maturation media container that has a liquid exchange port and contains a first maturation media that includes a ROCKi, an inhibitor of BMP signaling, and an inhibitor of GSK3β signaling. The automated cell culture system also includes a multiport valve that includes a valve actuator and: (a) a first selectable port, wherein the first selectable port is aseptically coupled to the first liquid exchange port of the first lid; and (b) a second selectable port, wherein the second selectable port is aseptically coupled to the liquid exchange port of the first maturation media container; (c) a fluid pump that comprises a pump actuator, wherein the fluid pump is coupled to the valve; and (d) a controller that is configured to actuate the valve actuator and the pump actuator. The controller: i) sends a first valve control signal to actuate the valve to open the first selectable port to place the first maturation media container in fluid communication with the first liquid exchange port of the first cell culture container, and ii) sends a first pump control signal to actuate the fluid pump and to move the first maturation media from the first maturation media container to the cell culture container.

In some embodiments, the method also involves using the automated cell culture system to perform a media exchange. In these embodiments, the multiport valve has at least a third selectable port that is coupled to a waste container, and the method further includes directing the controller to conduct a first media exchange by: i) sending a second valve control signal to actuate the valve to place the waste container in fluid communication with the first liquid exchange port of the first cell culture container; ii) sending a second pump control signal to actuate the fluid pump and to move the from the first cell culture container to the waste container; iii) sending a third valve control signal to actuate the valve to open the first selectable port to place the first maturation media container in fluid communication with the first liquid exchange port of the first cell culture container; and iv) sending a third pump control signal to actuate the fluid pump to move the first maturation media from the first maturation media container to the first cell culture container. In some embodiments, the controller directs the automated cell culture system to perform a media exchange on each of Days 0, 1, 2, and 3 of the second incubation by repeating these steps. In some embodiments, the first maturation media includes a ROCKi, an inhibitor of BMP signaling, and an inhibitor of GSK3β signaling.

In some embodiments, the method also involves using the automated cell culture system to perform a media exchange in which two different media are used. In these embodiments, the multiport valve further includes at least a third selectable port that is coupled to a waste container and a fourth selectable port that is coupled to a second maturation media container that contains a second maturation media, and the method further comprises directing the controller to conduct a second media exchange by: i) sending a second valve control signal to actuate the valve to place the waste container in fluid communication with the first liquid exchange port of the first cell culture container; ii) sending a second pump control signal to actuate the fluid pump and to move the media from the first cell culture container to the waste container; iii) sending a third valve control signal to actuate the valve to open the third selectable port to place the second maturation media container in fluid communication with the first liquid exchange port of the first cell culture container; and iv) send a third pump control signal to actuate the fluid pump to move the second maturation media from the second maturation media container to the first cell culture container. In some embodiments, the second maturation media includes an inhibitor of GSK3β signaling, BDNF, GDNF, ascorbic acid, dbcAMP and TGFβ3. In some embodiments, the media exchange is conducted on Day 4 of the second incubation. In some embodiments, the controller directs the automated cell culture system to conduct additional media exchanges in which fresh aliquots of the second maturation media are exchanged with the second maturation media that is in the cell culture container.

In some embodiments, the first incubation is performed using an automated cell culture system. In these embodiments, the first incubation involves adherent culture of the cells using an automated cell culture system that includes at least a first cell culture container configured to receive a sample therein, and a first lid coupled to the first cell culture container, wherein the first lid comprises a first liquid exchange port and a first gas exchange port. The system also has at least a first induction media container, wherein the first induction media container has a liquid exchange port and contains a first induction media that includes an inhibitor of TGF-β/activin-Nodal signaling and an inhibitor of bone morphogenetic protein (BMP) signaling. The cell culture system also includes a multiport valve that includes a valve actuator and: (a) a first selectable port, wherein the first selectable port is aseptically coupled to the first liquid exchange port of the first lid; and (b) a second selectable port, wherein the second selectable port is aseptically coupled to the liquid exchange port of the first induction media container. The automated cell culture system also includes a fluid pump that includes a pump actuator, wherein the fluid pump is coupled to the valve; and a controller that is configured to actuate the valve actuator and the pump actuator. In some embodiments, the controller: i) sends a first valve control signal to actuate the valve to open the first selectable port to place the first induction media container in fluid communication with the first liquid exchange port of the first cell culture container, and ii) sends a first pump control signal to actuate the fluid pump to move the media from the first induction media container to the cell culture container.

In some embodiments, the methods also involve using the automated cell culture system to perform a media exchange as described above, except that the media used for the first incubation differ from those described above for the first incubation. In some embodiments, a first media exchange in which the first induction media that includes an inhibitor of TGF-β/activin-Nodal signaling and an inhibitor of bone morphogenetic protein (BMP) signaling is replaced by a fresh aliquot of the first induction media. In some embodiments, the controller directs the automated cell culture system to perform this media exchange on one or more of Day 1 through Day 3 of the first incubation. In some embodiments, the second induction media that is used for the first incubation includes an activator of Sonic Hedgehog (SHH) signaling and the inhibitor of glycogen synthase kinase 3β (GSK3β) signaling. In these embodiments, the media exchange is performed on one or more of Days 1 through Day 6 of the first incubation. In some embodiments, the media exchange is performed on each of Days 1 through 6 of the first incubation.

Automated Differentiation of Cells from Pluripotent Stem Cells

The invention also provides methods for automated differentiation of pluripotent stem cells into desired differentiated cells. Differentiation of pluripotent stem cells into dopaminergic neuronal progenitor cells as described herein is one example of these methods, but the methods are also useful for obtaining other differentiated cells from PSCs. For example, the methods are useful for automated differentiation of pluripotent stem cells into stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells. See, e.g., U.S. patent application Ser. No. 18/135,069 entitled “METHODS OF CLASSIFYING THE DIFFERENTIATION STATE OF CELLS” for examples of differentiation protocols that are suitable for use with the automated differentiation methods described herein.

In some embodiments, the methods involve performing a first incubation in which pluripotent stem cells are non-adherently cultured in a cell culture bag, such as a multiwell cell culture bag, under conditions to produce a cellular spheroid of partially differentiated cells. The cells of the spheroid are then subjected to a second incubation to further differentiate the cells into the desired differentiated cell type. The second incubation is performed using an automated cell culture system.

As an example, the invention provides methods of differentiating pluripotent stem cells into dopaminergic neuronal progenitor cells. These methods involve performing a first incubation that includes non-adherently culturing pluripotent stem cells in a first cell culture bag under conditions to produce a cellular spheroid. The first incubation includes: (a) exposing the pluripotent stem cells to an inhibitor of TGF-β/activin-Nodal signaling and an inhibitor of bone morphogenetic protein (BMP) signaling for at least one day (Day 0); and (b) exposing the pluripotent stem cells to at least one activator of Sonic Hedgehog (SHH) signaling and an inhibitor of glycogen synthase kinase 3β (GSK3β) signaling.

The cells of the cellular spheroid are then subjected to a second incubation in which the cells are cultured in a second culture vessel under conditions to further differentiate the cells into dopaminergic neuronal progenitor cells. The second incubation is performed using an automated cell culture system that includes: (a) at least a first cell culture container configured to receive a sample therein, and a first lid coupled to the first cell culture container, wherein the first lid comprises a first liquid exchange port and a first gas exchange port; (b) at least a first maturation media container, wherein the first maturation media container comprises a liquid exchange port and contains a first maturation media that comprises a ROCKi, an inhibitor of BMP signaling, and an inhibitor of GSK3β signaling; (c) a multiport valve that includes a valve actuator, a first selectable port, wherein the first selectable port is aseptically coupled to the first liquid exchange port of the first lid and a second selectable port, wherein the second selectable port is aseptically coupled to the liquid exchange port of the first maturation media container; (d) a fluid pump that comprises a pump actuator, wherein the fluid pump is coupled to the valve; and (e) a controller that is configured to actuate the valve actuator and the pump actuator. The cells of the spheroid are placed in the first cell culture container and the controller: i) sends a first valve control signal to actuates the valve to open the first selectable port to place the first maturation media container in fluid communication with the first liquid exchange port of the first cell culture container, and ii) sends a first pump control signal to actuate the fluid pump and to move the first maturation media from the first maturation media container to the cell culture container. Suitable automated cell culture systems include those described in, for example, US Patent Application No. 2021/0238532.

In some embodiments, the first incubation of this fully automated dopaminergic neuronal progenitor cell differentiation protocol involves: (a) exposing the pluripotent stem cells to an inhibitor of TGF-β/activin-Nodal signaling and an inhibitor of bone morphogenetic protein (BMP) signaling for at least one day (Day 0); and (b) starting on the second day (Day 1) of the first incubation, exposing the pluripotent stem cells to at least one activator of Sonic Hedgehog (SHH) signaling and an inhibitor of glycogen synthase kinase 3β (GSK3β) signaling.

In some embodiments, the Day 0 incubation is done in the absence of: x) an activator of Sonic Hedgehog (SHH) signaling, and y) an inhibitor of glycogen synthase kinase 3β (GSK3β) signaling. In some embodiments, the method also includes exposing the pluripotent stem cells to a ROCK inhibitor starting on Day 0. In some embodiments, the pluripotent stem cells were not exposed to a ROCKi prior to exposing the pluripotent stem cells to the inhibitor of TGF-β/activin-Nodal signaling and the inhibitor of bone morphogenetic protein (BMP) signaling of the first incubation.

In some embodiments, the second incubation involves culturing cells of the spheroid in a second culture vessel that is coated with a substrate that promotes adhesion of the cells to the culture vessel. Beginning when the cells are placed in the second culture vessel, typically on Day 7, the cells are exposed to (i) an inhibitor of BMP signaling and (ii) an inhibitor of GSK3β signaling. In some embodiments, the second incubation also includes exposing the cells, typically on Day 11, to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

The use of a cell culture bag for the first phase of a differentiation protocol and an automated cell culture system for the second phase has significant advantages over previously known methods. For example, the use of automated systems decreases human-induced variability in the protocol, therefore yielding more consistent results. The reduction in human involvement also reduces the costs associated with producing the differentiated cells. Moreover, the combination of using a cell culture bag for the suspension culture part of the differentiation protocol and an automated cell culture system for the adherent part of the protocol yielded several times more differentiated dopaminergic neuronal progenitor cells than previously known methods.

### C. Compositions and Formulations

In embodiments of the provided methods, neutrally differentiated cells produced by the methods provided herein can be harvested or collected, such as for formulation and use of the cells. In some embodiments, the provided methods for producing differentiated cells, such as for use as a cell therapy in the treatment of a neurodegenerative disease may include formulation of cells, such as formulation of differentiated cells resulting from the provided methods described herein. In some embodiments, the dose of cells comprising differentiated cells (e.g., dopaminergic neuronal progenitor cells, including determined DA neuronal progenitor cells, committed dopaminergic neuronal progenitor cells, or dopaminergic neuronal cells), is provided as a composition or formulation, such as a pharmaceutical composition or formulation. Such compositions can be used in accord with the provided methods, such as in the prevention or treatment of neurodegenerative disorders, including Parkinson's disease.

In some embodiments, the differentiated cells in the provided therapeutic compositions, including those produced by any of the methods described herein, are capable of producing dopamine (DA). In some embodiments, the differentiated cells in the provided therapeutic compositions, including those produced by any of the methods described herein, do not produce or do not substantially produce norepinephrine (NE). Thus, in some embodiments, the differentiated cells in the therapeutic compositions provided herein, including those produced by any of the methods described herein, are capable of producing DA but do not produce or do not substantially produce NE. In some embodiments, the differentiated cells in the provided therapeutic compositions, including those produced by any of the methods described herein, do not produce or do not substantially produce serotonin. Thus, in some embodiments, the differentiated cells in the therapeutic compositions provided herein, including those produced by any of the methods described herein, are capable of producing DA but do not produce or do not substantially produce serotonin.

In some embodiments, the therapeutic compositions include dopaminergic neuronal progenitor cells that have one or more improved properties with respect to use as a treatment for a neurodegenerative disease such as Parkinson's disease, compared to neuronal cells produced using other differentiation methods, such as adherent culture differentiation methods. These improved properties can include, for example, one or more of the following: (a) expressing a higher level of FOXA2; (b) expressing a lower level of PAX6; (c) having a higher predicted graft size after implantation; (d) producing less serotonin; (e) including a higher percentage of viable cells; (f) expressing a higher level of CORIN; (g) expressing a lower level of PITX2; and (h) expressing a lower level of NKX2.1. In some embodiments, the dopaminergic neuronal progenitor cells in the therapeutic composition have two or more of these properties. In some embodiments, the cells have three or more of the properties, and in some embodiments, the cells have four, five, six, or seven or more of the properties. In some embodiments, the cells have all eight of the listed properties.

In some embodiments, the therapeutic compositions provided herein include dopaminergic neuronal progenitor cells that, compared to neuronal cells produced using an adherent culture differentiation method, exhibit one or more properties selected from the group consisting of: (a) expressing a higher level of FOXA2; (b) expressing a lower level of PAX6; (c) having a higher predicted graft size after implantation; (d) having a higher predicted dopamine production level after implantation; (e) producing less serotonin; (f) comprising a higher percentage of viable cells; (g) expressing a higher level of CORIN; (h) expressing a lower level of PITX2; and (i) expressing a lower level of NKX2.1. In some embodiments, the cells of the therapeutic compositions exhibit two or more of these properties. In some embodiments, the cells exhibit three, four, five, six, seven, or eight or more of these properties. In some embodiments, the cells exhibit all nine of the listed properties.

In some embodiments, the therapeutic compositions include dopaminergic neuronal progenitor cells that have a GraftTest score of at least 1,500, calculated as described in U.S. Provisional Application No. 63/598,533, entitled “METHODS OF PREDICTING CHARACTERISTICS OF DIFFERENTIATED NEURONAL CELLS,” filed Nov. 13, 2023. In some embodiments, the dopaminergic neuronal progenitor cells in the therapeutic compositions produce serotonin at a level that increases less than two-fold when stimulated with KCl compared to unstimulated baseline. In some embodiments, more than 99% of the dopaminergic neuronal progenitor cells in the therapeutic compositions are viable. In some embodiments, the therapeutic composition includes dopaminergic neuronal progenitor cells that express FOXA2 at greater than 100 TPM, based on bulk RNAseq analysis. In some embodiments, the dopaminergic neuronal progenitor cells in the therapeutic compositions express CORIN at greater than 300 TPM. In some embodiments, the dopaminergic neuronal progenitor cells in the therapeutic compositions express PITX2 at less than 50 TPM. In some embodiments, the dopaminergic neuronal progenitor cells in the therapeutic compositions express NKX2.1 at less than 10 TPM. In some embodiments, the dopaminergic neuronal progenitor cells in the therapeutic composition have two or more of these properties. In some embodiments, the cells have three or more of the properties, and in some embodiments, the cells have four, five, or six or more of the properties. In some embodiments, the cells have all seven of the properties listed in this paragraph.

In some cases, the cells are processed in one or more steps for manufacturing, generating or producing a cell therapy and/or differentiated cells may include formulation of cells, such as formulation of differentiated cells resulting from the methods. In some cases, the cells can be formulated in an amount for dosage administration, such as for a single unit dosage administration or multiple dosage administration.

In certain embodiments, one or more compositions of differentiated cells are formulated. In particular embodiments, one or more compositions of differentiated cells are formulated after the one or more compositions have been produced. In some embodiments, the one or more compositions have been previously cryopreserved and stored, and are thawed prior to the administration.

In certain embodiments, the differentiated cells include determined DA neuronal progenitor cells. In some embodiments, a formulated composition of differentiated cells is a composition enriched for determined DA neuronal progenitor cells. In certain embodiments, the differentiated cells include committed dopaminergic neuronal progenitor cells. In some embodiments, a formulated composition of differentiated cells is a composition enriched for committed dopaminergic neuronal progenitor cells. In certain embodiments, the differentiated cells include dopaminergic neuronal cells. In some embodiments, a formulated composition of differentiated cells is a composition enriched for dopaminergic neuronal cells.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are differentiated for at least 13 days. In some embodiments, the cells are differentiated for between 14 and 17 days. In some embodiments, the cells are differentiated for between 14 and 16 days. In some embodiments, the cells are differentiated for about 14 to 17 days. In some embodiments, the cells are differentiated for about 14 days. In some embodiments, the cells are differentiated for about 15 days. In some embodiments, the cells are differentiated for about 16 days. In some embodiments, the cells are differentiated for about 17 days.

In some embodiments, the cells are harvested after at least 13 days of culture. In some embodiments, the cells are harvested on between Day 14 and Day 17 of culture. In some embodiments, the cells are harvested on between Days 14 and 16 of culture. In some embodiments, the cells are harvested after about Day 13 of culture. In some embodiments, the cells are harvested after about Day 14 of culture. In some embodiments, the cells are harvested on about Day 14 of culture. In some embodiments, the cells are harvested on about Day 15 of culture. In some embodiments, the cells are harvested on about Day 16 of culture. In some embodiments, the cells are harvested on about Day 17 of culture.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the differentiated cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the neurodegenerative condition or disease (e.g., Parkinson's disease), such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cells are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the differentiated cells to replace the cells in a cryopreservative solution. In some embodiments, the cells are frozen, e.g., cryopreserved or cryoprotected, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of differentiated cells are formulated, cryopreserved, and then stored for an amount of time. In certain embodiments, the formulated, cryopreserved cells are stored until the cells are released for administration. In particular embodiments, the formulated cryopreserved cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryopreserved and stored for, for about, or for less than 1 days, 2 days, 3 days, 4 days, 5 days, 6 days, or 7 days. In certain embodiments, the cells are thawed and administered to a subject after the storage.

In some embodiments, the formulation is carried out using one or more processing step including washing, diluting or concentrating the cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to differentiated cells. In some embodiments, the volume of formulation buffer is from or from about 1 μL to 5000 μL, such as at least or about at least or about or 5 μL, 10 μL, 20 μL, 50 μL, 100 μL, 200 μL, 300 μL, 400 μL, 500 μL, 1000 μL, 2000 μL, 3000 μL, 4000 μL, or 5000 μL.

A container may generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, such cells produced by the method, or a composition comprising such cells, are administered to a subject for treating a neurodegenerative disease or condition.

### D. Methods of Treatment

Provided herein are methods of using any of the provided compositions for treating a disease or condition in a subject in need thereof. In particular embodiments, the composition is produced by the methods provided herein. Such methods and uses include therapeutic methods and uses, for example, involving administration of the therapeutic cells, or compositions containing the same, to a subject having a disease, condition, or disorder. In some embodiments the disease or condition is a neurodegenerative disease or condition. In some embodiments, the cells or pharmaceutical composition thereof is administered in an effective amount to effect treatment of the disease or disorder. Uses include uses of the cells or pharmaceutical compositions thereof in such methods and treatments, and in the preparation or manufacture of a medicament in order to carry out such therapeutic methods. In some embodiments, the methods thereby treat the disease or condition or disorder in the subject.

The present disclosure relates to methods of lineage specific differentiation of pluripotent stem cells (PSCs), including embryonic stem (ES) cells and induced pluripotent stem cells (iPSCs), for use in neurodegenerative diseases. Specifically, the methods, compositions, and uses thereof provided herein contemplate differentiation of pluripotent stem cells for administration to subjects exhibiting a loss of dopaminergic neuronal cells, including Parkinson's disease.

Parkinson's disease (PD) is the second most common neurodegenerative, estimated to affect 4-5 million patients worldwide. This number is predicted to more than double by 2030. PD is the second most common neurodegenerative disorder after Alzheimer's disease, affecting approximately 1 million patients in the US with 60,000 new patients diagnosed each year. Currently there is no cure for PD, which is characterized pathologically by a selective loss of midbrain DA neurons in the substantia nigra. A fundamental characteristic of PD is therefore progressive, severe and irreversible loss of midbrain dopaminergic neuronal cells resulting in ultimately disabling motor dysfunction.

In some embodiments, a subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [₁₈F]-L-DOPA PET scan. In some embodiments, the subject exhibits [₁₈F]-DG-PET evidence for a Parkinson's Disease Related Pattern (PDRP).

In some embodiments, the neurodegenerative disease is Parkinsonism. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has moderate or advanced Parkinson's disease. In some embodiments, the subject has mild Parkinson's disease. In some embodiments, the subject has a MDS-UPDRS motor score of between 33 and 60.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the striatum of the subject. In some embodiments, the dose of cells is administered to one hemisphere of the subject's striatum. In some embodiments, the dose of cells is administered to both hemispheres of the subject's.

In some embodiments, the dose of cells administered to the subject is about 5×10⁶ cells. In some embodiments, the dose of cells administered to the subject is about 10×10⁶ cells. In some embodiments, the dose of cells administered to the subject is about 15×10⁶ cells. In some embodiments, the dose of cells administered to the subject is about 20×10⁶ cells. In some embodiments, the dose of cells administered to the subject is about 25×10⁶ cells. In some embodiments, the dose of cells administered to the subject is about 30×10⁶ cells.

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about 3×10⁶ cells/hemisphere and 15×10⁶ cells/hemisphere. In some embodiments, the dose of cells is about 3×10⁶ cells/hemisphere. In some embodiments, the dose of cells is about 4×10⁶ cells/hemisphere. In some embodiments, the dose of cells is about 5×10⁶ cells/hemisphere. In some embodiments, the dose of cells is about about 6×10⁶ cells/hemisphere. In some embodiments, the dose of cells is about about 7×10⁶ cells/hemisphere. In some embodiments, the dose of cells is about about 8×10⁶ cells/hemisphere. In some embodiments, the dose of cells is about about 9×10⁶ cells/hemisphere. In some embodiments, the dose of cells is about 10×10⁶ cells/hemisphere. In some embodiments, the dose of cells is about 11×10⁶ cells/hemisphere. In some embodiments, the dose of cells is about 12×10⁶ cells/hemisphere. In some embodiments, the dose of cells is about 13×10⁶ cells/hemisphere. In some embodiments, the dose of cells is about 14×10⁶ cells/hemisphere. In some embodiments, the dose of cells is about 15×10⁶ cells/hemisphere.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g., determined dopaminergic neuronal progenitor cells or committed dopaminergic neuronal progenitor cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some embodiments, the cells are administered at a desired dosage, which in some aspects includes a desired dose or number of cells or cell type(s) and/or a desired ratio of cell types. Thus, the dosage of cells in some embodiments is based on a total number of cells and a desired ratio of the individual populations or sub-types. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some aspects, the size of the dose is determined based on one or more criteria such as response of the subject to prior treatment, e.g., disease type and/or stage, and/or likelihood or incidence of the subject developing toxic outcomes, e.g., dyskinesia.

### E. Articles of Manufacture

The invention also provides automated cell culture systems that are useful for differentiating pluripotent stem cells into dopaminergic neuronal progenitor cells. In some embodiments, the automated systems include: (a) at least a first cell culture container configured to receive a sample therein, and a first lid coupled to the first cell culture container, wherein the first lid comprises a first liquid exchange port and a first gas exchange port; (b) at least a first media container, wherein the first media container comprises a liquid exchange port, and (c) a valve that comprises: (i) a first selectable port, wherein the first selectable port is aseptically coupled to the first liquid exchange port of the first lid; and (i) a second selectable port, wherein the second selectable port is aseptically coupled to the liquid exchange port of the media container; and (d) a fluid pump coupled to the valve; wherein the first media container contains a first media that comprises an inhibitor of TGF-β/activin-Nodal signaling and an inhibitor of bone morphogenetic protein (BMP) signaling, and the fluid pump and the valve are configured to move the first media from the first media container to the first cell culture container.

In some embodiments, the automated culture systems further include a second media container that comprises a liquid exchange port, and the valve includes a third selectable port that is aseptically coupled to the liquid exchange port of the second media container, wherein the second media container contains a second media that includes an activator of Sonic Hedgehog (SHH) signaling and an inhibitor of glycogen synthase kinase 3β (GSK3β) signaling, and the fluid pump and the valve are configured to move the second media from the second media container to the first cell culture container. In some embodiments, the first media does not include an activator of Sonic Hedgehog (SHH) signaling or an inhibitor of glycogen synthase kinase 3β (GSK3β).

### EXAMPLES

The following examples are included for illustrative purposes only and are not intended to limit the scope of the invention.

### Example 1: Neuronal Differentiation of iPSCs

Induced pluripotent stem cells (iPSCs) were created from fibroblasts obtained from human donors with Parkinson's disease and subjected to a dopaminergic neuronal differentiation protocol using a manual process as described in U.S. patent application Ser. No. 18/742,917, filed Jun. 13, 2024.

iPSCs from the human donors were maintained by plating in Laminin-511 E8-coated 6-well plates. The cells were cultured without feeder cells in mTeSR™1-based media until they reached approximately 75-90% confluence. The iPSCs were then washed with sterile PBS and detached from the 6-well plates by enzymatic dissociation with Accutase®. The collected iPSCs were then used in the subsequent differentiation protocol.

The collected iPSCs were re-suspended in “basal induction media” (see Table E1 below) and seeded under non-adherent conditions using 24-well AggreWell™ plates. The iPSCs were seeded at 3.6×10⁶ cells/well at day 0, with approximately 3,000 cells/microwell in media supplemented as described below for each of the enumerated days of the differentiation method, e.g., Day 0 through Day 16. The cells were cultured for 7 days under non-adherent conditions, with media replacement as detailed below, to form spheroids. On Day 7, the resulting spheroids were dissociated into single cells by enzymatic dissociation with AccuMAX™, and the cells were plated as monolayers at a concentration of 800,000 cells/cm² on plates coated with a Laminin-511 E8 fragment for the remainder of culture, and further supplemented with nutrients and small molecules as described below.

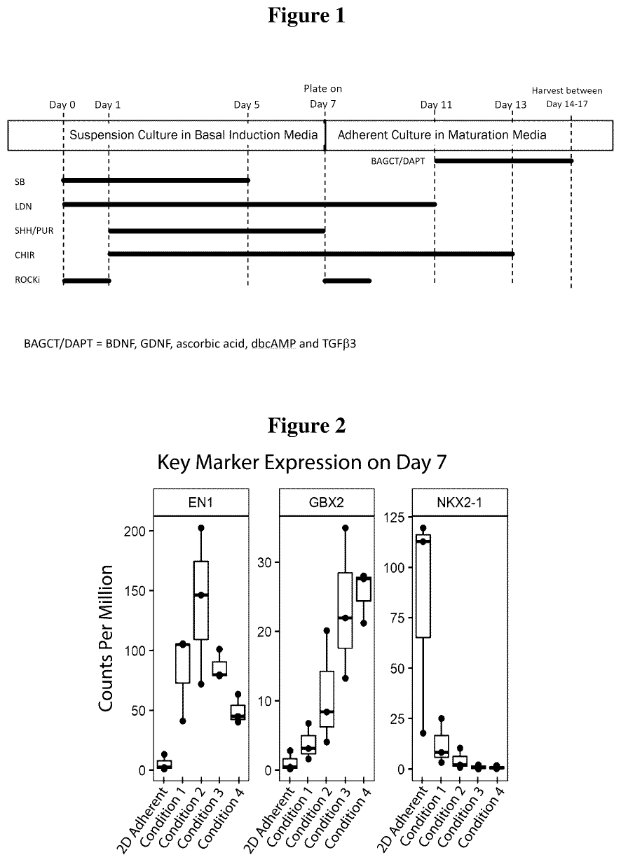

A schematic of the exemplary non-adherent differentiation protocol is shown in FIG. 1 and Table 1, which depict the small molecule compounds that were added at various days during the differentiation method. From Days 0 to 10, cells were cultured in the basal induction media, which was formulated to contain Neurobasal™ media and DMEM/F12 media at a 1:1 ratio (and with N-2 and B27 supplements, non-essential amino acids (NEAA), GlutaMAX™, L-glutamine, β-mercaptoethanol, and insulin), and supplemented with the appropriate small molecule compound(s). From Day 11 up to harvest, e.g., at Day 16, cells were cultured in a “maturation media” (Neurobasal™ media containing N-2 and B27 supplements, non-essential amino acids (NEAA), and GlutaMAX™), and supplemented with the appropriate small molecule compound(s). The basal induction media also included a serum replacement.

TABLE 1
Differentiation Protocol

| Day | Media | Small Molecules | | | | | | | |
|---|---|---|---|---|---|---|---|---|---|
| 0* | Basal | 5% S | LDN | SB | | | | ROCKi | |
| | Induction | | | | | | | | |
| 1 | Basal | 5% S | LDN | SB | SHH | PUR | CHIR | | |
| | Induction | | | | | | | | |
| 2 | Basal | 2% S | LDN | SB | SHH | PUR | CHIR | | |
| | Induction | | | | | | | | |
| 3 | Basal | 2% S | LDN | SB | SHH | PUR | CHIR | | |
| | Induction | | | | | | | | |
| 4 | Basal | 2% S | LDN | SB | SHH | PUR | CHIR | | |
| | Induction | | | | | | | | |
| 5 | Basal | 2% S | LDN | | SHH | PUR | CHIR | | |
| | Induction | | | | | | | | |
| 6 | Basal | 2% S | LDN | | SHH | PUR | CHIR | | |
| | Induction | | | | | | | | |
| 7* | Basal | 2% S | LDN | | | | CHIR | ROCKi | |
| | Induction | | | | | | | | |
| 8 | Basal | 2% S | LDN | | | | CHIR | | |
| | Induction | | | | | | | | |
| 9 | Basal | 2% S | LDN | | | | CHIR | | |
| | Induction | | | | | | | | |
| 10 | Basal | 2% S | LDN | | | | CHIR | | |
| | Induction | | | | | | | | |
| 11 | Maturation | | BDNF | GDNF | ascorbic | dbcAMP | CHIR | TGFβ3 | DAPT |
| 12 | Maturation | | BDNF | GDNF | ascorbic | dbcAMP | CHIR | TGFβ3 | DAPT |
| 13 | Maturation | | BDNF | GDNF | ascorbic | dbcAMP | | TGFβ3 | DAPT |
| 14 | Maturation | | BDNF | GDNF | ascorbic | dbcAMP | | TGFβ3 | DAPT |
| 15 | Maturation | | BDNF | GDNF | ascorbic | dbcAMP | | TGFβ3 | DAPT |
| 16 | Maturation | | BDNF | GDNF | ascorbic | dbcAMP | | TGFβ3 | DAPT |
| 17 | Maturation | | BDNF | GDNF | ascorbic | dbcAMP | | TGFβ3 | DAPT |

S: Serum replacement;
LDN: LDN193189;
SB: SB431542;
SHH: recombinant mouse Sonic Hedgehog (rmSHH);
PUR: Purmorphamine;
CHIR: CHIR99021;
ROCKi: Y-27632;
BDNF: recombinant human brain-derived neurotrophic factor (rhBDNF);
GDNF: recombinant human glial cell-derived neurotrophic factor (rhGDNF);
TGFβ3: recombinant human transforming growth factor beta 3 (rhTGFβ3);
dbcAMP: dibutyryl cyclic AMP;
Ascorbic: ascorbic acid;
*Indicates media supplemented with ROCK inhibitor (Y-27632)

On Day 0, the basal induction media was formulated to contain: 5% serum replacement, 0.1 μM LDN (1×), 10 μM SB (1×), and 10 μM of the ROCK inhibitor Y-27632. On Day 1, basal induction media was formulated to contain: 5% serum replacement, 0.2 μM LDN (2×), 20 μM SB (2×), 0.2 μg/mL SHH (2×), 4 μM PUR (2×), and 2 μM of the GSK3β inhibitor CHIR99021, and was added by a 50% media exchange. Since the basal induction media added on Days 1 through 6 were added by a daily 50% media exchange, the concentrations of small molecules LDN, SHH, and PUR in the basal induction media were doubled (2×) on Days 1 to 6 as compared to if a complete replacement of media was performed, and the concentration of small molecule SB was doubled (2×) on Days 1 to 4, as compared to if a complete replacement of media was performed. Also, the basal induction media on Days 2 to 6 was formulated to contain 2% serum replacement and 4 μM CHIR99021. Accordingly, the basal induction media formulated for Days 2 to 4 contained 2% serum replacement, 0.2 μM LDN, 20 μM SB, 0.2 μg/mL SHH, 4 μM PUR, and 4 μM CHIR99021, and the basal induction media formulated for Days 5 and 6 contained 2% serum replacement, 0.2 μM LDN, 0.2 μg/mL SHH, 4 μM PUR, and 4 μM CHIR99021.

The cells were transferred to substrate-coated plates on Day 7, as described above, using a basal induction media that was formulated to contain: 2% serum replacement, 0.1 μM LDN, 2 μM CHIR99021, and 10 μM Y-27632. The media was completely replaced daily from Days 8 to 10, with basal induction media formulated to contain 2% serum replacement, 0.1 μM LDN, and 2 μM CHIR99021.

Starting on Day 11, the media was switched to maturation media formulated to contain: 20 ng/mL BDNF, 0.2 mM ascorbic acid, 20 ng/mL GDNF, 0.5 mM dbcAMP, and 1 ng/mL TGFβ3 (collectively, “BAGCT”), 10 μM DAPT, and 2 μM CHIR99021. The media was completely replaced on Day 12 with the same media formulation containing the same concentrations of small molecule compounds as on Day 11. From Day 13 until harvest, the media was replaced every Day with maturation media formulated to contain BAGCT and DAPT (collectively, “BAGCT/DAPT”) at the same concentrations as on Days 11 and 12.

Cells were harvested on Day 16.

### Example 2: Comparative Analysis of Neuronal Differentiation Methods

The non-automated neuronal differentiation method as described in Example 1 was compared with an adherent neuronal differentiation method and three alternative non-adherent neuronal differentiation methods. iPSCs were created from fibroblasts obtained from human donors with Parkinson's disease and subjected to each of these five neuronal differentiation methods through at least Day 10 for comparative purposes.

The adherent neuronal differentiation method (also referred to herein as “21D adherent culture”) involved culturing the iPSCs in mTeSR1 based media until they reached approximately 75-90% confluence. The iPSCs were then washed with sterile PBS and detached from the 6-well plates by enzymatic dissociation with Accutase®. On Day −1, the cells were seeded at approximately 300,000 cells/cm² with 10 μM of the ROCK inhibitor Y-27632 overnight, with 2.9e⁶ cells per well of a 6-well plate in mTeSR1 based media. The cells were then cultured in a basal induction media containing the components as shown in Table 2, below, beginning on Day 0 for each of the enumerated days through Day 10. The media was completed replaced each of Days 0 through 10 with the media as shown in Table E2. The basal induction media contained, when indicated: LDN at a concentration of 0.1 μM LDN; SB at a concentration of 10 μM SB; SHH at a concentration of 0.1 μg/mL; and PUR at a concentration of 2 μM.

TABLE 2
2D Adherent Culture

| Day | Media | Small Molecules | | | | | | |
|---|---|---|---|---|---|---|---|---|
| −1 | mTeSR1 | | | | | | | ROCKi |
| 0 | Basal | 5% S | LDN | SB | | | | |
| | Induction | | | | | | | |
| 1 | Basal | 5% S | LDN | SB | SHH | PUR | | |
| | Induction | | | | | | | |
| 2 | Basal | 2% S | LDN | SB | SHH | PUR | CHIR | |
| | Induction | | | | | | | |
| 3 | Basal | 2% S | LDN | SB | SHH | PUR | CHIR | |
| | Induction | | | | | | | |
| 4 | Basal | 2% S | LDN | SB | SHH | PUR | CHIR | |
| | Induction | | | | | | | |
| 5 | Basal | 2% S | LDN | | SHH | PUR | CHIR | |
| | Induction | | | | | | | |
| 6 | Basal | 2% S | LDN | | SHH | PUR | CHIR | |
| | Induction | | | | | | | |
| 7 | Basal | 2% S | LDN | | | | CHIR | |
| | Induction | | | | | | | |
| 8 | Basal | 2% S | LDN | | | | CHIR | |
| | Induction | | | | | | | |
| 9 | Basal | 2% S | LDN | | | | CHIR | |
| | Induction | | | | | | | |
| 10 | Basal | 2% S | LDN | | | | CHIR | |
| | Induction | | | | | | | |

S: Serum replacement;
LDN: LDN193189;
SB: SB431542;
SHH: recombinant mouse Sonic Hedgehog (rmSHH);
PUR: Purmorphamine;
CHIR: CHIR99021;
ROCKi: Y-27632

The non-adherent neuronal differentiation method as described in Example 1 (also referred to as “3D Condition 2”) was performed in addition to three alternative non-adherent methods (referred to as “3D Condition 1,” “3D Condition 3,” and 3D Condition 4”) that were each performed using the same method as 3D Condition 2 except for the differences as indicated in Table E3, below. Specifically, each of 3D Conditions 1, 2, 3, and 4 are the same except for differences in the small molecules contained in the basal induction media on Day 0 and Day 1. The basal induction media of Day 2 and beyond for each of 3D Conditions 1, 2, 3, and 4 was the same, thereby resulting in the same methodology and media used except for Days 0 and 1, as shown in Table 3 below. In particular, the basal induction media of 3D Condition 1 prepared for Day 1 did not contain CHIR until Day 2, whereas the basal induction media of 3D Conditions 2 and 3 prepared for Day 1 each contained CHIR at a different concentration (2 μM for 3D Condition 2; and 4 μM for 3D Condition 3). The basal induction media for 3D Condition 4 that was prepared for Day 0 further included SHH, PUR, and CHIR at the indicated concentrations. 3D Condition 4 is the non-adherent neuronal differentiation method as described in Example 1 of WO2021/146349.

TABLE 3
Conditions of D 0 and D 1 Basal Induction
Media for 3D Conditions 1, 2, 3, and 4

| Condition | Day 0 | Day 1 |
|---|---|---|
| 3D Condition 1 | 5% serum replacement | 5% serum replacement |
| | LDN (0.1 μM) | LDN (0.2 μM) |
| | SB (10 μM) | SB (20 μM) |
| | | SHH (0.2 μg/mL) |
| | | PUR (4 μM) |
| 3D Condition 2 | 5% serum replacement | 5% serum replacement |
| | LDN (0.1 μM) | LDN (0.2 μM) |
| | SB (10 μM) | SB (20 μM) |
| | | SHH (0.2 μg/mL) |
| | | PUR (4 μM) |
| | | CHIR (2 μM) |
| 3D Condition 3 | 5% serum replacement | 5% serum replacement |
| | LDN (0.1 μM) | LDN (0.2 μM) |
| | SB (10 μM) | SB (20 μM) |
| | | SHH (0.2 μg/mL) |
| | | PUR (4 μM) |
| | | CHIR (4 μM) |
| 3D Condition 4 | 5% serum replacement | 5% serum replacement |
| | LDN (0.1 μM) | LDN (0.2 μM) |
| | SB (10 μM) | SB (20 μM) |
| | SHH (0.1 μg/mL) | SHH (0.2 μg/mL) |
| | PUR (2 μM) | PUR (4 μM) |
| | CHIR (2 μM) | CHIR (4 μM) |

Cells were differentiated in accordance with each of the five differentiation methods and were analyzed at days including Day 7 and Day 10 for expression of various markers.

EN1 is a midbrain dopaminergic lineage marker. Expression levels of the EN1 dopaminergic lineage marker were compared between cells generated from each of the five differentiation methods at Day 7. As shown in FIG. 2, the cells cultured using the manual 2D adherent culture method exhibited very low EN1 expression on Day 7, and the lowest among all five methods tested, whereas the cells cultured using 3D Condition 2 exhibited the highest expression of EN1 on Day 7 of all five differentiation methods, and was nearly three times higher on average than when the cells were cultured with 3D Condition 4.

GBX2 and NKX2-1 are off-target non-dopaminergic lineage markers. As shown in FIG. 2, on Day 7, expression of the GBX2 marker is relatively low for all five differentiation methods, as the average counts per million is below 30 for each of the methods, and expression of the NKX2-1 marker is also very low (under 30 counts per million) for all four non-adherent methods (3D Conditions 1, 2, 3, and 4). Surprisingly, the non-adherent methods (3D Conditions 1, 2, 3, and 4) exhibit considerably less NKX2-1 marker expression on Day 7 than the 2D adherent culture method (approximately 10-fold less on average), thereby demonstrating the superior ability of the non-adherent methods, including 3D Condition 2, to preferentially promote a dopaminergic lineage fate.

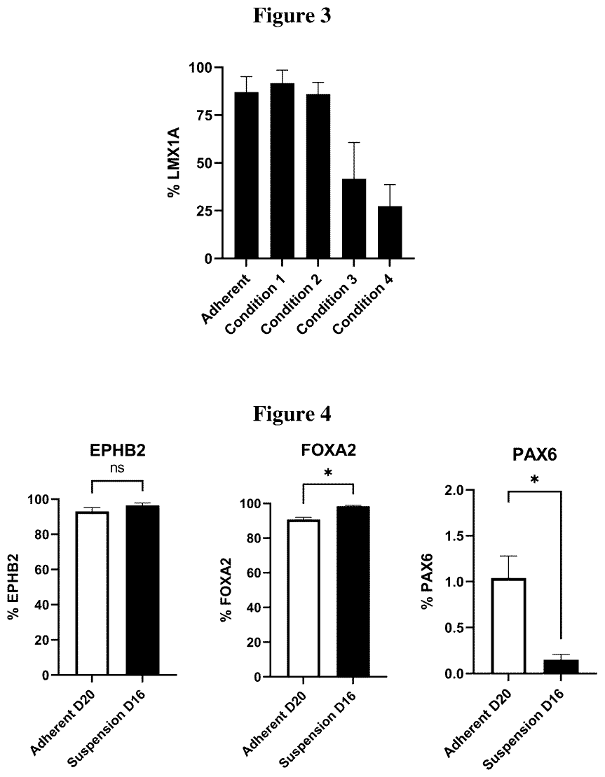

LMX1A is a ventral midbrain marker indicative of dopaminergic lineage fate. As shown in FIG. 3A and FIG. 3B, on Day 10, expression of LMX1A in cells cultured in 3D Conditions 1 and 2 was similar to expression of LMX1A in cells cultured in the 2D adherent culture, which were all higher than the expression of LMX1A in cells cultured in 3D Conditions 3 and 4.

Collectively, this data demonstrates that 3D Condition 2 is superior with regards to promoting a dopaminergic lineage fate early on in the differentiation process, as compared to the 2D adherent culture method and the three alternative non-adherent methods (3D Conditions 1, 3, and 4), which includes 3D Condition 4 in which the cells are exposed to LDN, SB, SHH, PUR, and CHIR beginning on Day 0.

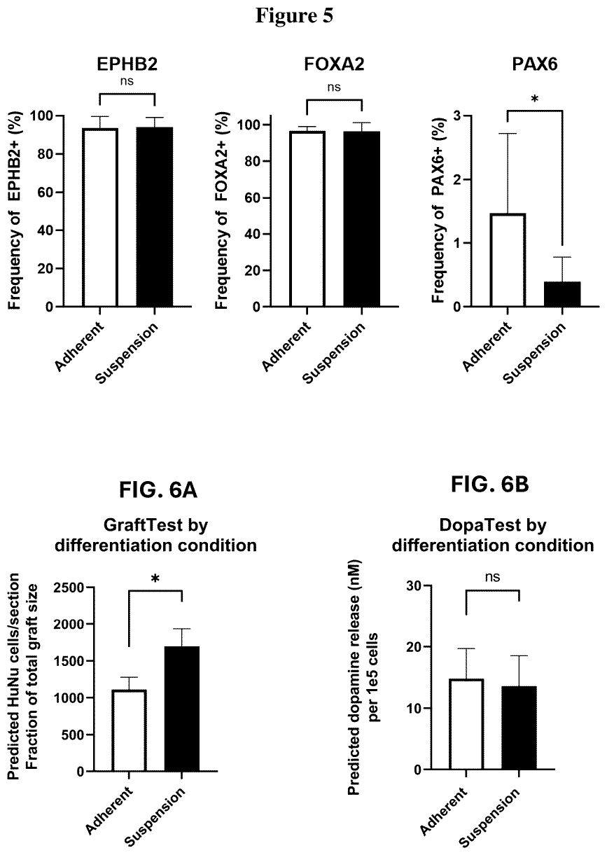

FIG. 4 shows a comparison of marker expression by neuronal cells produced using adherent cell culture versus the Day 1 suspension culture protocol described herein. The Day 1 suspension culture cells were harvested on Day 16, while the adherent cultured cells were harvested on Day 20. FIG. 4A shows expression of the pan-neuronal marker EPHB2, which is indicative of the neuronal cell lineage in general. Similar levels of EPHB2 were expressed by neuronal cells obtained using adherent and suspension culture, indicating that both differentiation protocols produce neuronal progenitor cells. FIG. 4B shows expression of FOXA2, which is characteristic of the floorplate lineage in brain development. The desired dopaminergic neuronal progenitor cells are of the floorplate lineage. Neuronal cells obtained using the cell suspension culture method exhibited higher FOXA2 expression than neuronal cells produced using adherent culture, thereby demonstrating that the suspension culture differentiation protocol more accurately directs cells towards the desired ventral midbrain dopaminergic neuronal cell fate. FIG. 4C shows a comparison of PAX6 expression by cells produced using adherent culture versus cells produced using the suspension culture protocol. PAX6 expression of the forebrain lineage in brain development. Cells produced using the suspension culture differentiation method exhibited an approximately 10-fold decrease in PAX6 expression compared to cells produced using adherent culture, thereby providing further confirmation that that the suspension culture differentiation method more accurately directs differentiation towards the desired ventral midbrain dopaminergic neuronal cell fate. FIG. 5 shows corresponding marker expression for DANPCs produced using the automated adherent and suspension culture protocols described herein.

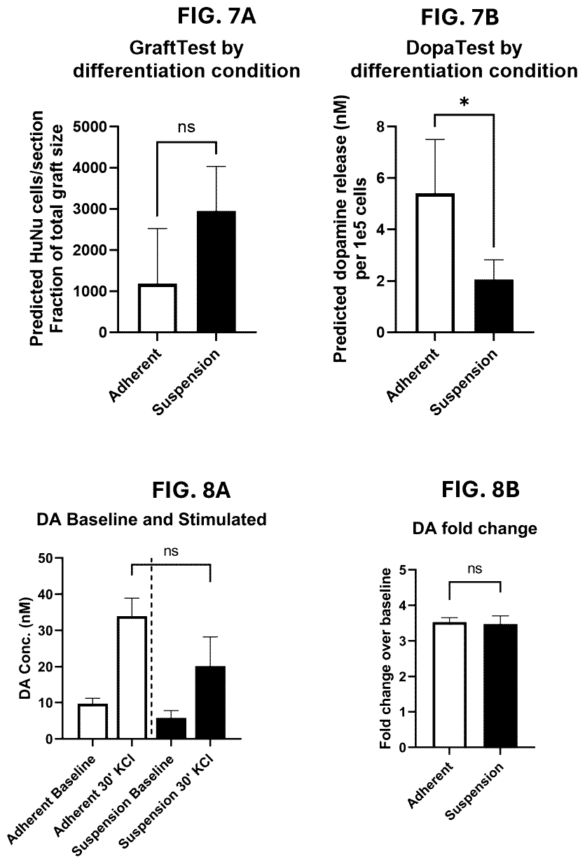

Cells obtained using the “Day 1” suspension culture differentiation method described herein were then compared to cells produced using the adherent culture differentiation protocol for predicted graft size and dopamine production after the cells are implanted into a subject brain. The Day 1 suspension culture cells were harvested on Day 16, while the adherent cultured cells were harvested on Day 20. Predicted graft sizes were obtained GraftTest™, which is an in silico predictive tool that uses bulk RNAseq data to estimate graft size (number of human nuclei in a rodent brain hemisphere). GraftTest is described in US Patent Publ. No. US2025154606A1 entitled “METHODS OF PREDICTING ENGRAFTMENT CAPABILITY OF DIFFERENTIATED NEURONAL CELLS,” published May 15, 2025. The model was trained on graft features (human nuclei) that were measured after the precursor cells had engrafted and matured in the rodent brain for 21 days. GraftTest scores were obtained from a model that was trained on cell counts found in one sixth of the total graft volume in each hemisphere. Each estimate represents the mean of several hemispheres. Training data typically included estimates based on four to five rodents and two hemispheres per rodent. FIG. 6A shows that dopaminergic neuronal progenitor cells produced using the manual suspension culture differentiation method are predicted to produce significantly larger grafts after implantation than neuronal progenitor cells produced using the adherent culture differentiation method. FIG. 7A shows GraftTest results for DANPCs produced using automated culture protocols.

Predicted dopamine release levels after implantation for cells produced using the manual Day 1 suspension culture differentiation method compared to cells produced using the adherent culture differentiation protocol are shown in FIG. 6B. The Day 1 suspension culture cells were harvested on Day 16, while the adherent cultured cells were harvested on Day 20. The predicted dopamine release levels were obtained using DopaTest™, which is an in silico predictive tool that uses bulk RNAseq data to estimate the quantity of dopamine released by cells after extended culture. DopaTest is described in US Patent Publ. No. US2025157580A1 entitled “METHODS OF PREDICTING DOPAMINE PRODUCTION CAPABILITY OF DIFFERENTIATED NEURONAL CELLS,” published May 15, 2025. In the training data set, dopamine was quantified using Liquid Chromatography Mass Spectrometry (LCMS) and transcriptome features at the precursor focal timepoint were used to train the model. Predictions of dopamine release therefore represent a measure of expected cell performance after extended culture. Dopamine release estimates are based on predicted dopamine production (nM) of 1e5 mature dopaminergic neuron cells. As shown in FIG. 6B, predicted dopamine release levels were not significantly different for cells produced using Day 1 suspension culture versus cells produced using the adherent culture method. FIG. 7B shows corresponding DopaTest results for DANPCs produced using automated culture protocols.

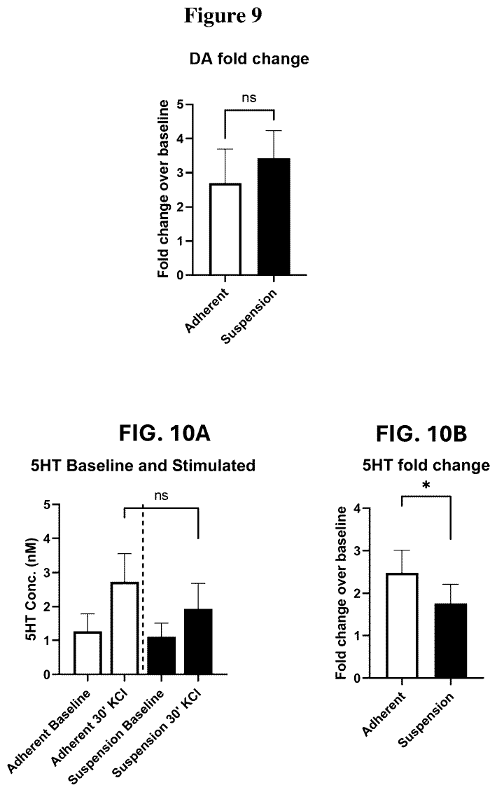

Liquid Chromatography Mass Spectrometry (LCMS) was used to compare dopamine and serotonin release by cells obtained using the Day 1 suspension culture differentiation method were compared to those produced using adherent culture. The Day 1 suspension culture cells were harvested on Day 16, while the adherent cultured cells were harvested on Day 20. Cells were then cultured for 60 days in a defined neuronal maturation media. On the day of collection, cells were rinsed once with warm Hank's balanced salt solution (HBSS), then incubated in HBSS for 15 minutes at 37 deg. Celsius. Samples were collected after 15 minutes in triplicates to establish baseline neurotransmitter release. Cells were then incubated at 37 deg Celsius in a 56 mM potassium chloride (KCl) solution in HBSS to induce neurotransmitter release. Samples were collected in triplicate at the 30′ timepoint. FIG. 8A shows dopamine concentration of the supernatant (nM) at baseline and after a 30 minute KCl treatment for both manual adherent and suspension culture protocols, without normalization (n=4). There was no significant difference in the raw dopamine release between differentiation conditions (n.s. p>0.05). FIG. 8B shows the fold change of the dopamine release upon stimulation to dopamine release at baseline. There was no significant difference in the raw dopamine release between the adherent and suspension culture protocols (n.s. p>0.05). Error bars are shown as the standard error of the mean (S.E.M.). n=4 for all plots. FIG. 9 shows the fold change for dopamine production by DANPCs produced using automated protocols as described herein.

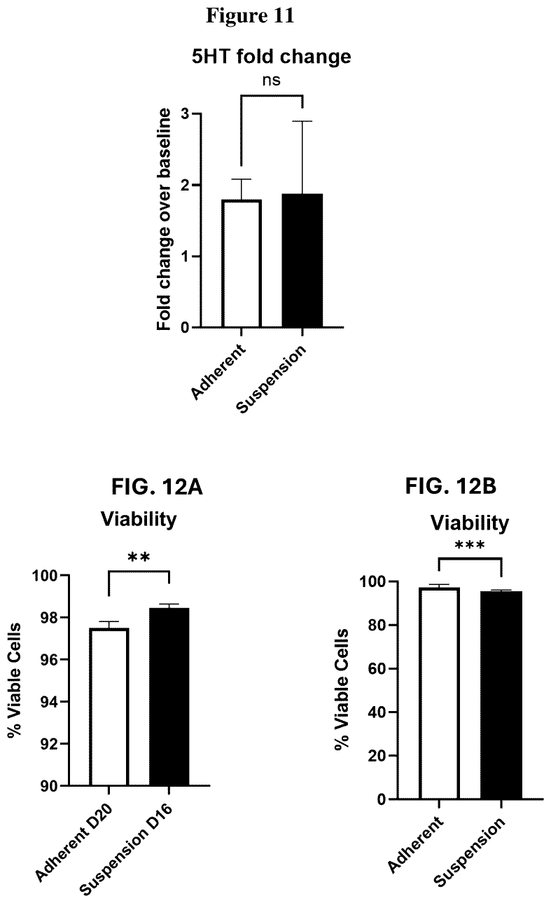

FIG. 10A shows serotonin concentration of the supernatant (nM) at baseline and after a 30-minute KCl treatment for both manual adherent and suspension culture protocols, without normalization (n=4). There was no significant difference in the raw serotonin release between the adherent and suspension culture protocols (n.s. p>0.05). FIG. 10B shows normalization of serotonin release upon KCl stimulation compared to baseline. The data show that there is a significant decrease in serotonin (5HT) production by cells produced using the Day 1 suspension culture differentiation method compared to cells produced using the adherent culture differentiation method (n=4)(* p<0.05). Error bars are shown as the standard error of the mean (S.E.M.). These results demonstrate that the Day 1 suspension culture differentiation method yields cells that have an enhanced safety profile compared to cells produced using adherent cell culture. FIG. 11 shows serotonin fold change for DANPCs produced using the automated protocols described herein.

We then compared viability of cells produced using the Day 1 suspension culture differentiation method to that of cells produced using the adherent cell culture differentiation method. The Day 1 suspension culture cells were harvested on Day 16, while the adherent cultured cells were harvested on Day 20. Cell viability was tested by running the “cell count and viability” protocol on the Nucleocounter NC-200. Cells were generated either using the adherent or suspension culture conditions (n=4). Cells generated using the manual Day 1 suspension culture condition showed a significant increase in percentage of viable cells as shown in FIG. 12A. DANPCs produced using the automated protocols described herein also had high viability (FIG. 12B).

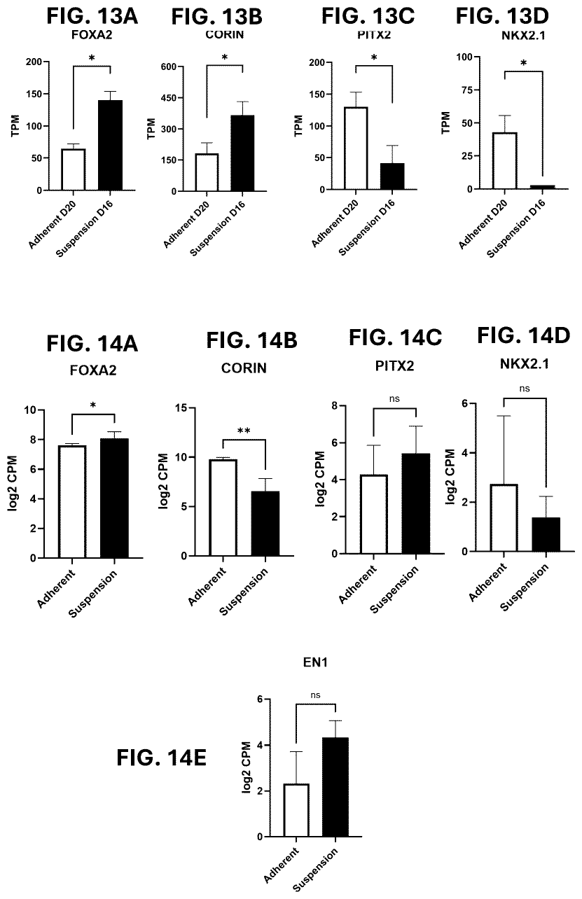

Cells produced using the manual Day 1 suspension culture differentiation method were compared to those produced using adherent cell culture differentiation with respect to expression of two “on-target” markers and two “off-target” markers. The Day 1 suspension culture cells were harvested on Day 16, while the adherent cultured cells were harvested on Day 20. RNAseq analysis shows that cells produced using the Day 1 suspension culture method exhibited higher levels of “on target” FOXA2 (FIG. 13A) and CORIN expression (FIG. 13B) than cells produced using the adherent method, thereby indicating that the Day 1 suspension culture cells are more characteristic of the desired dopaminergic neuronal progenitor cells than are cells produced using adherent culture. Cells produced using the Day 1 suspension culture method also expressed significantly less of the “off-target” markers PITX2 (FIG. 13C) and NKX2.1 (FIG. 13D), providing further evidence that the Day 1 suspension culture differentiation method produces cells that are more accurately directed to the desired dopaminergic neuronal progenitor cell type. FIG. 14A-E provide corresponding expression levels for DANPCs produced using automated protocols as described herein.

### Example 3: Differentiation of iPSCs into Dopaminergic Neuronal Progenitor Cells Using Suspension Culture Protocol in Cell Culture Bag

Prior to Day 0, iPSCs were expanded manually and were grown on laminin 511e8 plates, fed daily with mTESR. The day prior to Day 0, 15 mL DMEMF12 was added. On Day 0, any bubbles were removed from the microwells within the cell culture bag (“Wellbag”) prior to introducing the cells to the culture bag. Bubbles were removed by adding 15 mL of fresh Day 0 media supplemented with ROCK inhibitor (Table 1). The bag was then inspected under a microscope to ensure that all bubbles were removed from the microwells. If not, the process was repeated until all bubbles were removed.

Once all bubbles were removed, iPSCs were then dissociated to single cells with Accutase©, counted on a NucleoCounter, and seeded into a Wellbag containing 18,000 microwells at a concentration of 500 cells per microwell, for a total of 9 million cells per Wellbag on Day 0. This was accomplished by making a cell solution of 1.8 million cells per mL in Day 0 media+Rocki and adding 5 mL of this cell suspension to the Wellbag. The bag was gently agitated to evenly distribute all cells, and then the Wellbag was placed into the pressure plate and the lid was tightly screwed in, ensuring there were no creases in the bag. The bag was allowed to rest on a flat surface at room temperature for 5-10 minutes before placing into a 37° C. incubator w/ 95% humidity and 5% CO₂ for 24 hours. After 24+/−1 hour, the first media exchange was initiated.

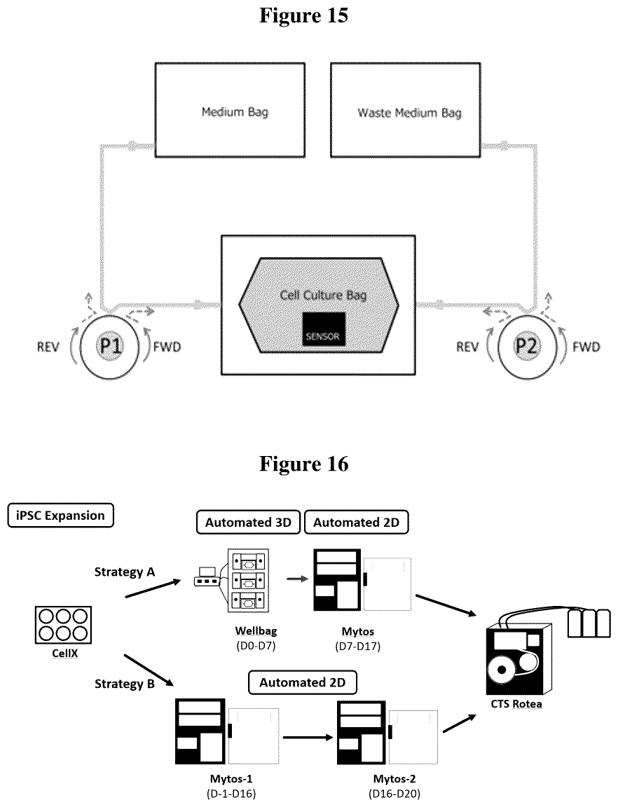

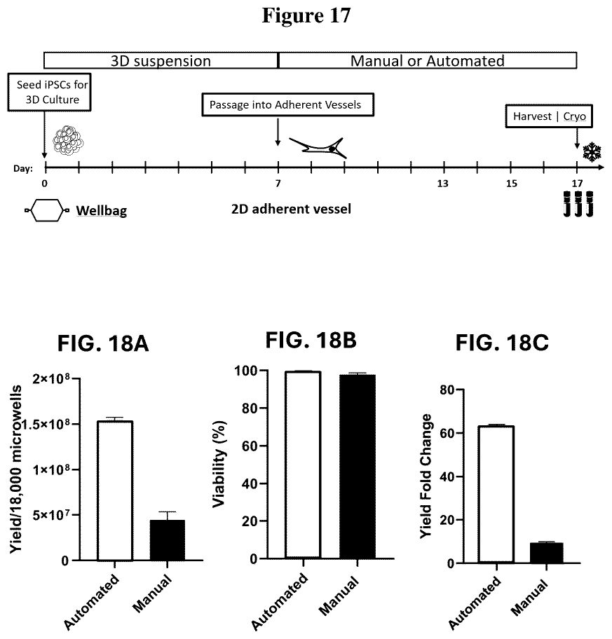

On each of Days 1-6, a media exchange was performed. First 25 mL of air and 30 mL of fresh Day 1 media were injected into a media bag. The inlet port of pump 1 was attached to this bag, and the outlet port of pump 1 was attached to the cell culture bag that contained the cells. An empty bag was connected to the inlet port of pump 2 to act as a waste bag. The pumps were then run to feed the newly formed spheroids (FIG. 15). This was repeated daily—replacing the media bag with fresh media of the corresponding day of culture, emptying out the waste bag, and running the pumps—until Day 7.

On Day 7, the lid of the pressing plate was unscrewed and the Wellbag was removed and gently agitated to bring the spheroids into suspension. A 50 mL syringe was attached to one end of the bag and 15 mL air was injected into the Wellbag before gently collecting the spheroids in suspension. This suspension was passed through a 37 μm reversible cell strainer. The Wellbag was rinsed once with 20 mL of wash medium, the resulting spheroid suspension was collected and passed through the same strainer. After all spheroids had been collected from the Wellbag, the 37 μm strainer was rinsed with 10 mL of 1×PBS without calcium and without magnesium (“PBS (−)(−).” The cell strainer was then inverted into a new sterile 50 mL conical tube and the spheroids dislodged with 20 mL of 1×PBS (−)(−). The 50 mL conical tube was spun in a centrifuge set at 200 g for 2 min. The PBS was gently aspirated without dislodging the spheroids at the bottom of the tube. Then, 20 mL of AccuMax was added to the conical tube, which was gently agitated to dislodge the spheroids. The conical tube was placed in a bead bath set at 37° C. for 20 minutes. After 10 minutes, the spheroids were gently triturated with a 10 mL serological pipette until spheroids had become single cells.

As shown in FIG. 18A, performing the suspension culture differentiation protocol in cell culture bags provided a significantly higher yield of differentiated cells after a seven day first incubation than previously available methods in which the suspension culture was conducted using microwell plates. Cell viability was similar for the cell culture bag and the microwell plate methods FIG. 18B). FIG. 18C shows that that the yield of dopaminergic neuronal progenitor cells was several fold higher when the differentiation protocol was performed using a microwell cell culture bag than using a manual process that used multiwell plates.

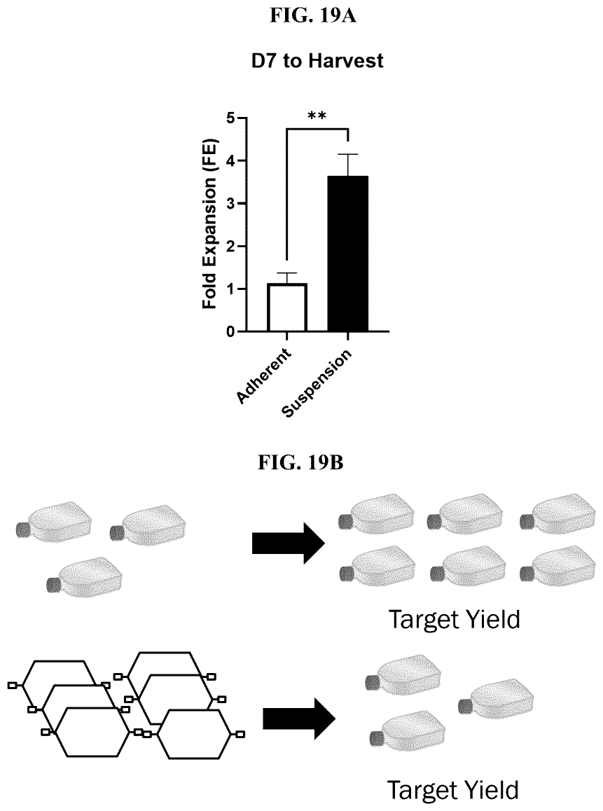

FIG. 19A shows the fold expansion (FE) of DANPCs from Day 7 to harvest for the adherent versus automated suspension culture protocols. Both Wellbag and the Elplasia suspension culture protocols yielded several times more DANPCs than the adherent protocols. FIG. 19B shows a comparison of the number of culture vessels required to obtain 1.2e9 DANPCs for manual adherent culture (top) versus automated suspension culture (bottom). The automated suspension culture protocol produced the desired number of DANPCs using only half the number of culture vessels, greatly increasing the efficiency of the manufacturing process.

### Example 4: Differentiation of iPSCs into Dopaminergic Neuronal Progenitor Cells Using Automated Cell Culture System Using Cell Culture Bag

This Example describes the use of a Mytos automated cell culture system for the adherent phase of the dopaminergic neuronal progenitor cell differentiation protocol after the initiation phase of the differentiation protocol was performed in suspension culture as described in Example 4.

The flasks in the Mytos automated cell culture device were first coated with laminin 511-e8. To do this, each flask received 30-35 mL of 1.25 μg/mL laminin 511-e8 in 1×PBS (−)(−). Flasks were left to incubate at 37° C. for 2 hours. After 2 hours, the laminin coating solution was diluted out by doing 3 serial dilutions with Day 7 media+ROCKi. This results in a solution of Day 7 media+ROCKi with less than 10% laminin/PBS solution. The culture vessel was attached to the Mytos automated cell culture device.

The cells obtained as described in Example 4 were suspended in Day 7 media+ROCKi, counted, and the cell culture device added 20 ml of cell suspension to each of three cell culture vessels at a concentration of 7 million cells per mL plated onto the 511e8 coated cell culture vessel, which provided a seeding density of 800,000 cells per cm² of the cell culture vessel. The device gently agitated the flasks in a swirling motion to evenly distribute the cells within the flask. The cells then settled and adhered to the flask. The device took 100 images per flask 2 hours and 22 hours after seeding. Twenty-four hours after the seeding of the cells, the device performed the first media exchange in which it aspirated 40 mL of spent media and add 35 mL of Fresh Day 8 media.

On Days 8-16, the device continued imaging 100 times, twice daily and the cell cultures were automatically fed after the media connection was made. To perform the media change, the device aspirated spent media and added fresh media daily until Day 16.

On Day 16, the cells were ready for dissociation. The device aspirated all spent media from each flask and rinsed each flask with 20 mL of 1×PBS (−)(−) twice. After rinsing, the device aspirated all 1×PBS and added 15 mL of Accutase® to each flask. After one hour the device added an additional 10 mL of Accutase®. The cells were incubated in Accutase® for roughly 1.5 hours until the cells appeared ready for dissociation to single cells. Once the cells were ready, the device shook the flask vigorously and then triturated the cells 2× through its pump system. The device then combined the cell suspensions of all 3 flasks into one collection vessel. The cells were then manually counted on a Nucleocounter, spun down in a 50 mL conical tube at 600 g for 5 min, resuspended in cryoprotectant, added to cryovials, and froze down in a controlled rate freezer.

### Example 5: Differentiation of iPSCs into Dopaminergic Neuronal Progenitor Cells Using Automated Cell Culture System

This Example describes the use of a Mytos automated cell culture system for the complete dopaminergic neuronal progenitor cell differentiation protocol, conducted entirely using adherent cell culture.

The flasks in the Mytos automated cell culture device were first coated with laminin 511-e8. To do this, each flask received 30-35 mL of 1.25 μg/mL laminin 511-e8 in 1×PBS (−)(−). Flasks were left to incubate at 37° C. for 2 hours. After 2 hours, the laminin coating solution was diluted out by doing 3 serial dilutions with Day 7 media+ROCKi. This resulted in a solution of Day 7 media+ROCKi with less than 10% laminin/PBS solution. The culture vessel was then attached to the Mytos automated cell culture device.

Prior to Day −1, iPSCs were expanded manually and were grown on laminin 511e8 plates, fed daily with mTESR. On Day −1, iPSCs were dissociated to single cell using Accutase®, spun down in a centrifuge in a 50 mL conical tube at 600 g for 5 min, resuspended in mTESR+ROCKi, counted on a NucleoCounter, and then loaded into the seeding vessel on the Mytos device at a concentration of 7.4e+06 cells/mL. Then 7 mL of cells were added to each flask for a final concentration of 0.296e+06 cells/cm². The device gently agitated the flasks in a swirling motion to evenly distribute the cells within the flask. The cells then settled and adhered to the flask. The device then took 100 images per flask 2 hours and 22 hours after seeding. 24 hours after the seeding of the cells, the device performed the first media exchange in which it aspirated 30-35 mL of spent media and add 30-35 mL of fresh Day 0 media. The device then aspirated and added fresh media daily until Day 16, and take 100 images per flask twice daily.

On Day 16, the device coated the remaining flasks as described above. On Day 16, the cells were ready for dissociation. The device aspirated all spent media from each flask and rinsed each flask with 20 ml of 1×PBS (−)(−) twice. After rinsing, the device aspirated all 1×PBS and added 15 mL of Accutase® to each flask. The cells were incubated in Accutase® for roughly 1.5 hours until the cells appeared ready for dissociation to single cells. Once the cells were ready, the device shook the flask vigorously and then triturated the cells twice through its pump system. The device then combined the cell suspensions of both flasks into one collection flask.

The device washed the collection flasks with Day 16 media+ROCKi. The device then distributed the cells that were collected from the two flasks into four freshly coated flasks (completing a 1:2 passage) with Day 16 media+ROCKi. The device gently agitated the flasks in a swirling motion to evenly distribute the cells within the flask. The cells then settled and adhered to the flask. The device took 100 images per flask 2 hours and 22 hours after seeding. 24 hours after the seeding of the cells, the device performed a media exchange in which it aspirated 30-35 mL of spent media and added 30-35 mL of fresh Day 17 media. The device then aspirated and added fresh media on Day 19, and take 100 images per flask twice daily.

On Day 20, the cells were ready for dissociation. The device aspirated all spent media from each flask and rinsed each flask with 20 mL of 1×PBS (−)(−) twice. After rinsing, the device aspirated all 1×PBS and add 25 mL of Accutase® to each flask. The cells will incubate in Accutase® for roughly 1.5 hours until the cells appeared ready for dissociation to single cells. Once the cells were ready, the device shook the flask vigorously and then triturated the cells 2× through its pump system. The device then combined the cell suspensions of all 4 flasks into one collection flask. The cells were then manually counted on a NucleoCounter, spun down in a 50 mL conical tube at 600 g for 5 min, resuspended in cryoprotectant, added to cryovials, and froze down in a controlled rate freezer.

### Example 6: Characterization of Cells Produced Using Automated Differentiation Protocol

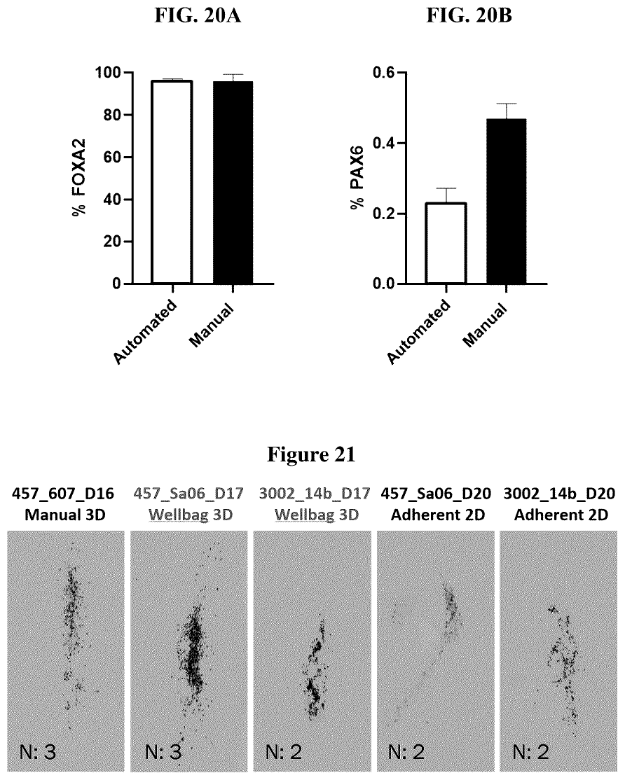

Key metrics for a DANPC manufacturing process include the percentage of samples that meet a desired threshold for an on-target marker, and that do not exceed a desired threshold for expression of an off-target marker. A and B show that cell populations obtained using the automated suspension culture differentiation method described herein have equivalent levels of the on-target marker FOXA2 compared to cell populations obtained using manual differentiation methods (FIG. 20A) while the percentage of cells that express the off-target marker PAX6 is significantly reduced when cells are differentiated using the automated suspension culture method (FIG. 20B)

To evaluate whether PAX6 failure is a function of the donor phenotype or the manufacturing process, we used the automated suspension culture differentiation protocol to differentiate iPSC cell lines that, when differentiated using a manual adherent manufacturing process, failed quality control due to PAX6 expression exceeding the threshold. As shown in Table 4 below, the iPSC cell lines 04_003c and 04_009d failed the PAX6 test when differentiated using an adherent differentiation protocol with harvesting at 20 days. When these same two cell lines were differentiated using an automated suspension culture differentiation protocol as described herein, the resulting DANPCs passed the PAX6 test (Table 5). This result demonstrates that the automated 3D suspension culture manufacturing protocol can overcome PAX6 failures. PAX6 pass or fail is therefore not predetermined for particular donors.

TABLE 4
Adherent 20 Day Protocol

| | Flow Cytometry | | | | |
|---|---|---|---|---|---|
| Marker | % FOXA2⁺ | % OTX2⁺ | % EPHB2⁺ | % KI67⁺ | % PAX6⁺ |
| Pass/Fail Threshold | >80% | >80% | >80% | | <5% |
| Clinical Historical Data | 95 | 87 | 98 | 27 | 3 |
| 04_003c | 94.88 | 97.9 | 76.77 | 39.43 | 11.37 |
| | | | | | (fail) |
| 04_009d | 97.59 | 94.44 | 94.37 | 26.63 | 8.92 |
| | | | | | (fail) |

TABLE 5
Wellbag 17 Day Protocol

| | Flow Cytometry | | | | |
|---|---|---|---|---|---|
| Marker | % FOXA2⁺ | % OTX2⁺ | % EPHB2⁺ | % KI67⁺ | % PAX6⁺ |
| Pass/Fail Threshold | >80% | >80% | >80% | | <5% |
| Clinical Historical Data | 95 | 87 | 98 | 27 | 3 |
| 04_003c | 98.8 | 94.9 | 96.2 | 32.3 | 0.12 |
| | | | | | (pass) |
| 04_009d | 98.5 | 94.3 | 96.4 | 41.3 | 0.12 |
| | | | | | (pass) |

### Example 7: In Vivo Engraftment of DANPCs

DANPCs obtained using the automated 3D (Wellbag) and manual differentiation protocols were implanted into rat brains and graft sizes were determined. As shown in FIG. 21, both methods yielded DANPCs that successfully engraft and survive at least 21 days when implanted into rat brains, shows that graft sizes correlated well with the predicted engraftment capability as determined by GraftTest. Nuclei counts and GraftTest results for these samples, which are shown in Table 6, confirm the effectiveness of the automated suspension culture protocol described herein for manufacturing DANPCs.

TABLE 6

| Group | Test Article | Nuclei Counts | GraftTest |
|---|---|---|---|
| 1 | 457_607_D16 | 208-1127 | 2155 |
| | Manual 3D | | |
| 2 | 457_Sa06_D17 | 524-2566 | 12104 |
| | Wellbag 3D | | |
| 3 | 3002_Sa06_D17 | 495-1554 | 3823 |
| | Wellbag 3D | | |
| 4 | 457_Sa06_D20 | 33-287 | 4125 |
| | Adherent 2D | | |
| 5 | 3002_14b_D20 | 24-530 | 828 |
| | Adherent 2D | | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of differentiating pluripotent stem cells into dopaminergic neuronal progenitor cells, the method comprising:
a) performing a first incubation comprising non-adherently culturing pluripotent stem cells in a cell culture bag under conditions that promote cellular spheroid formation, wherein the first incubation comprises:
i) exposing the pluripotent stem cells to a first medium that comprises an inhibitor of TGF-β/activin-Nodal signaling and an inhibitor of bone morphogenetic protein (BMP) signaling; and
ii) subsequently exposing the resulting partially differentiated cells to a second medium that comprises an activator of Sonic Hedgehog (SHH) signaling and an inhibitor of glycogen synthase kinase 3β (GSK3β) signaling; and
b) transferring the spheroids to a second culture vessel and culturing them under adherent conditions to promote differentiation into dopaminergic neuronal progenitor cells.

2. The method of claim 1, wherein a mechanical or pneumatic pressure is applied to the cell culture bag during the first incubation.

3. The method of claim 1, wherein the cell culture bag comprises a plurality of wells or recesses on at least one surface of the cell culture bag.

4. The method of claim 1, wherein the cell culture bag comprises at least one port, and is operably connected to a fluidic system comprising: a) a first pump configured to deliver the second medium from a media bag into the cell culture bag; and b) a second pump configured to remove the first medium from the cell culture bag into a waste bag, wherein the pumps are operated to effect a media exchange.

5. The method of claim 4, wherein the second pump is operated to remove all or a portion of the first medium from the cell culture bag prior to operation of the first pump to deliver the second medium into the cell culture bag.

6. The method of claim 4, wherein the first pump and the second pump are operated simultaneously during at least a portion of the media exchange cycle.

7. The method of claim 4, wherein the media exchange is regulated by a control system comprising: a) a voltage sensor configured to detect medium thickness or volume within the cell culture bag or a holder unit; and b) a computer system executing software instructions to control the operation of the first and second pumps based on input from the voltage sensor.

8. The method of claim 1, wherein a media exchange is performed on each of Days 1 through Day 6 of the first incubation.

9. The method of claim 1, wherein the method further comprises agitating the cell culture bag during the first incubation to bring the cellular spheroids into suspension.

10. The method of claim 1, further comprising collecting the cellular spheroids.

11. The method of claim 1, wherein the method comprises:
a) exposing the pluripotent stem cells to the first medium for at least one day (Day 0) in the absence of: x) an activator of Sonic Hedgehog (SHH) signaling, and y) an inhibitor of glycogen synthase kinase 3β (GSK3β) signaling; and
b) starting on the second day (Day 1) of the first incubation, exposing the cells to the second medium that comprises the activator of Sonic Hedgehog (SHH) signaling and the inhibitor of glycogen synthase kinase 3β (GSK3β) signaling.

12. The method of claim 1, wherein the second incubation is performed using an automated cell culture system.

13. The method of claim 1, wherein the second culture vessel comprises a multiwell plate or a tissue culture flask.

14. The method of claim 13, wherein the second culture vessel is compatible with an automated cell culture system.

15. The method of claim 14, wherein the second culture vessel is selected based on compatibility with a Mytos automation platform.

16. A method of differentiating pluripotent stem cells into dopaminergic neuronal progenitor cells, the method comprising:
a) performing a first incubation comprising culturing pluripotent stem cells in a first culture vessel, wherein the first incubation comprises:
i) exposing the pluripotent stem cells to a first medium that comprises an inhibitor of TGF-β/activin-Nodal signaling and an inhibitor of bone morphogenetic protein (BMP) signaling; and
ii) subsequently exposing the resultant partially differentiated cells to a second medium that comprises at least one activator of Sonic Hedgehog (SHH) signaling and an inhibitor of glycogen synthase kinase 3β (GSK3β) signaling; and
b) performing a second incubation comprising adherently culturing the cells in a second culture vessel under conditions that promote differentiation into dopaminergic neuronal progenitor cells;
wherein at least one of the first incubation and the second incubation is performed using an automated cell culture system.

17. The method of claim 16, wherein the automated cell culture system comprises:
a) a first cell culture container configured to receive a sample therein, and a first lid coupled to the first cell culture container, wherein the first lid comprises a first liquid exchange port and a first gas exchange port;
b) a first maturation media container comprising a liquid exchange port and containing a first maturation medium that comprises a ROCK inhibitor (ROCKi), an inhibitor of BMP signaling, and an inhibitor of GSK3β signaling;
c) a multiport valve comprising a valve actuator and:
i) a first selectable port, wherein the first selectable port is coupled to the first liquid exchange port of the first lid; and
ii) a second selectable port coupled to the liquid exchange port of the first maturation media container;
d) a fluid pump comprising a pump actuator, wherein the fluid pump is coupled to the valve; and
e) a controller configured to actuate the valve actuator and the pump actuator; wherein the controller: i) sends a first valve control signal to actuate the valve to open the first selectable port to place the first maturation media container in fluid communication with the first liquid exchange port of the first cell culture container, and ii) sends a first pump control signal to actuate the fluid pump to move the first maturation media from the first maturation media container to the cell culture container.

18. The method of claim 17, wherein the automated cell culture system further comprises:
a) a waste container; and
b) at least one upstream or downstream valve configured to selectively place the waste container or the first maturation media container in fluid communication with the first liquid exchange port of the first cell culture container; and the method further comprises directing the controller to perform a media exchange by:
i) actuating the at least one valve to place the waste container in fluid communication with the first liquid exchange port of the first cell culture container;
ii) actuating the fluid pump to move medium from the first cell culture container to the waste container;
iii) actuating the at least one valve to place the first maturation media container in fluid communication with the first liquid exchange port of the first cell culture container; and
iv) actuating the fluid pump to move the first maturation medium from the first maturation media container to the first cell culture container.

19. The method of claim 17, wherein the media exchange is conducted on each of Days 0, 1, 2, and 3 of the second incubation.

20. The method of claim 17, wherein the automated cell culture system further comprises:
a) a second maturation media container comprising a liquid exchange port and containing a second maturation medium; and
b) at least one upstream or downstream valve configured to selectively place the second maturation media container in fluid communication with the first liquid exchange port of the first cell culture container; and wherein the controller is further configured to perform a second media exchange by:
i) actuating the at least one valve to place the waste container in fluid communication with the first liquid exchange port of the first cell culture container;
ii) actuating the fluid pump to move medium from the first cell culture container to the waste container;
iii) actuating the at least one valve to place the second maturation media container in fluid communication with the first liquid exchange port of the first cell culture container; and
iv) actuating the fluid pump to move the second maturation medium from the second maturation media container to the first cell culture container.

21. The method of claim 20, wherein the second maturation media comprises an inhibitor of GSK3β signaling, BDNF, GDNF, ascorbic acid, dbcAMP and TGFβ3.

22. The method of claim 20, wherein the second media exchange is conducted on Day 4 of the second incubation.

23. The method of claim 16, wherein the first incubation is performed using an automated cell culture system that comprises:
a) a first cell culture container configured to receive a sample therein, and a first lid coupled to the first cell culture container, wherein the first lid comprises a first liquid exchange port and a first gas exchange port;
b) a first induction media container comprising a liquid exchange port and containing a first induction medium that comprises an inhibitor of TGF-β/activin-Nodal signaling and an inhibitor of bone morphogenetic protein (BMP) signaling;
c) a multiport valve comprising a valve actuator that comprises:
i) a first selectable port, wherein the first selectable port is coupled to the first liquid exchange port of the first lid; and
ii) a second selectable port, wherein the second selectable port is aseptically coupled to the liquid exchange port of the first induction media container; and
d) a fluid pump comprising a pump actuator, wherein the fluid pump is coupled to the valve; and
e) a controller configured to actuate the valve actuator and the pump actuator;
f) wherein the controller;
wherein the controller: i) sends a first valve control signal to actuate the valve to open the first selectable port to place the first induction media container in fluid communication with the first liquid exchange port of the first cell culture container; and ii) sends a first pump control signal to actuate the fluid pump to move the first induction medium from the first induction media container to the first cell culture container.

24. The method of claim 23 wherein the automated cell culture system further comprises:
a) a waste container; and
b) at least one upstream or downstream valve configured to selectively place the waste container or the first induction media container in fluid communication with the first liquid exchange port of the first cell culture container; and
wherein the controller is further configured to perform a first media exchange by:
(i) actuating the at least one valve to place the waste container in fluid communication with the first liquid exchange port of the first cell culture container;
(ii) actuating the fluid pump to move medium from the first cell culture container to the waste container;
(iii) actuating the at least one valve to place the first induction media container in fluid communication with the first liquid exchange port of the first cell culture container; and
(iv) actuating the fluid pump to move the first induction medium from the first induction media container to the first cell culture container.

25. The method of claim 24, wherein the first media exchange is conducted on one or more of Day 1 through Day 3 of the first incubation.

26. The method of claim 23, wherein the automated cell culture system further comprises:
a) a second induction media container comprising a liquid exchange port and containing a second induction medium that comprises an activator of Sonic Hedgehog (SHH) signaling and an inhibitor of glycogen synthase kinase 3β (GSK3β) signaling; and
b) at least one upstream or downstream valve configured to selectively place the second induction media container in fluid communication with the first liquid exchange port of the first cell culture container;
c) and wherein the controller is further configured to perform a second media exchange by: i) actuating the at least one valve to place the waste container in fluid communication with the first liquid exchange port of the first cell culture container; ii) actuating the fluid pump to move medium from the first cell culture container to the waste container; iii) ctuating the at least one valve to place the second induction media container in fluid communication with the first liquid exchange port of the first cell culture container; and iv actuating the fluid pump to move the second induction medium from the second induction media container to the first cell culture container.

27. The method of claim 26, wherein the second media exchange is conducted on one or more of Day 1 through Day 6 of the first incubation.

28. An automated cell culture system comprising:
a) at least a first cell culture container configured to receive a sample therein, and a first lid coupled to the first cell culture container, wherein the first lid comprises a first liquid exchange port and a first gas exchange port;
b) at least a first media container, wherein the first media container comprises a liquid exchange port, and
c) a valve that comprises:
i) a first selectable port, wherein the first selectable port is aseptically coupled to the first liquid exchange port of the first lid; and
ii) a second selectable port, wherein the second selectable port is aseptically coupled to the liquid exchange port of the media container; and
d) a fluid pump coupled to the valve;
wherein the first media container contains a first media that comprises an inhibitor of TGF-β/activin-Nodal signaling and an inhibitor of bone morphogenetic protein (BMP) signaling, and the fluid pump and the valve are configured to move the first media from the first media container to the first cell culture container.

29. The automated cell culture system of claim 28, wherein the system further comprises a second media container that comprises a liquid exchange port, and the valve comprises a third selectable port that is aseptically coupled to the liquid exchange port of the second media container, wherein the second media container contains a second media that comprises an activator of Sonic Hedgehog (SHH) signaling and an inhibitor of glycogen synthase kinase 3β (GSK3β) signaling, and the fluid pump and the valve are configured to move the second media from the second media container to the first cell culture container.

30. The automated cell culture system of claim 28, wherein the first media does not include an activator of Sonic Hedgehog (SHH) signaling or an inhibitor of glycogen synthase kinase 3β (GSK3β).

31. A method of differentiating pluripotent stem cells into dopaminergic neuronal progenitor cells, the method comprising:
a) performing a first incubation comprising non-adherently culturing pluripotent stem cells in a first cell culture bag under conditions that promote cellular spheroid formation, wherein the first incubation comprises:
i) exposing the pluripotent stem cells to an inhibitor of TGF-β/activin-Nodal signaling and an inhibitor of bone morphogenetic protein (BMP) signaling; and
ii) subsequently exposing the pluripotent stem cells to at least one activator of Sonic Hedgehog (SHH) signaling and an inhibitor of glycogen synthase kinase 3β (GSK3β) signaling; and
b) performing a second incubation comprising adherently culturing cells of the spheroid in a second culture vessel under conditions to further differentiate the cells into dopaminergic neuronal progenitor cells; wherein the second incubation is performed using an automated cell culture system that comprises:
i) a first cell culture container configured to receive a sample therein, and a first lid coupled to the first cell culture container, wherein the first lid comprises a first liquid exchange port and a first gas exchange port;
ii) a first maturation media container, wherein the first maturation media container comprises a liquid exchange port and contains a first maturation media that comprises a ROCKi, an inhibitor of BMP signaling, and an inhibitor of GSK3β signaling;
iii) a multiport valve that comprises a valve actuator and:
1. a first selectable port of the multiport valve coupled to the first liquid exchange port of the first lid; and
2. a second selectable port of the multiport valve coupled to the liquid exchange port of the first maturation media container;
iv) a fluid pump comprising a pump actuator, wherein the fluid pump is coupled to the multiport valve; and
v) a controller configured to actuate the valve actuator and the pump actuator;
wherein the cells of the spheroid are placed in the first cell culture container and the controller: i) sends a first valve control signal to actuate the valve to open the first selectable port to place the first maturation media container in fluid communication with the first liquid exchange port of the first cell culture container, and ii) sends a first pump control signal to actuate the fluid pump to move the first maturation medium from the first maturation media container to the first cell culture container.

32. The method of claim 31, wherein the first incubation comprises:
a) exposing the pluripotent stem cells to the first medium for at least one day (Day 0), in the absence of: x) an activator of Sonic Hedgehog (SHH) signaling, and y) an inhibitor of glycogen synthase kinase 3β (GSK3β) signaling; and
b) starting on the second day (Day 1) of the first incubation, exposing the cells to the second medium comprising the activator of SHH signaling and the inhibitor of GSK3β signaling.

33. The method of claim 1, wherein the dopaminergic neuronal progenitor cells are determined dopaminergic neuronal progenitor cells.

34. The method of claim 1, wherein the pluripotent stem cells are induced pluripotent stem cells.

35. The method of claim 1, wherein the pluripotent stem cells are autologous to a subject to be treated with the dopaminergic neuronal progenitor cells.

36. The method of claim 1, wherein the second incubation begins on or about Day 7.

37. The method of claim 1, wherein the first incubation further comprises exposing the pluripotent stem cells to a ROCK inhibitor (ROCKi) starting on Day 0.

38. The method of claim 37, wherein the pluripotent stem cells were not exposed to a ROCKi prior to exposing the pluripotent stem cells to the inhibitor of TGF-β/activin-Nodal signaling and the inhibitor of bone morphogenetic protein (BMP) signaling of the first incubation.

39. The method of claim 1, wherein the inhibitor of BMP signaling is LDN193189.

40. The method of claim 39, wherein the cells are exposed to LDN193189 at a concentration of between about 10 nM and 500 nM, between about 20 nM and about 400 nM, between about 50 nM and about 200 nM, or between about 75 nM and about 150 nM, optionally about 100 nM.

41. The method of claim 1, wherein the inhibitor of TGF-β/activin-Nodal signaling is SB431542.

42. The method of claim 41, wherein the cells are exposed to SB431542 at a concentration of between about 1 μM and about 20 μM, between about 5 μM and about 15 μM, or between about 8 μM and about 12 μM, optionally about 10 μM.

43. The method of claim 1, wherein the activator of SHH signaling is SHH or purmorphamine.

44. The method of claim 43, wherein the cells are exposed to SHH at a concentration of between about 10 ng/mL and 500 ng/mL, between about 20 ng/mL and about 400 ng/mL, between about 50 ng/mL and about 200 ng/mL, or between about 75 ng/mL and about 150 ng/mL, optionally about 100 ng/mL.

45. The method of claim 43, wherein the cells are exposed to purmorphamine at a concentration of between about 0.1 μM and about 20 μM, between about 0.5 μM and about 10 μM, between about 1 μM and about 5 μM, between about 1 μM and about 3 μM, or between about 1.5 μM and about 2.5 μM, optionally at about 2 μM.

46. The method of claim 1, wherein the inhibitor of GSK3β signaling is CHIR99021.

47. The method of claim 1, wherein the first incubation comprises a media exchange on one or more of Days 1 through 6.

48. The method of claim 47, wherein the first incubation comprises a media exchange on each of Days 1 through 6.

49. The method of claim 1, wherein the second incubation comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

50. The method of claim 1, wherein the method further comprises harvesting the dopaminergic neuronal progenitor cells.

51. The method of claim 50, wherein the dopaminergic neuronal progenitor cells are harvested on Day 14 or later.

52. The method of claim 50, wherein the dopaminergic neuronal progenitor cells are harvested between Day 14 and Day 17.

53. The method of claim 50, wherein the method further comprises formulating the harvested dopaminergic neuronal progenitor cells with a cryoprotectant.

54. The method of claim 53, wherein the method further comprises cryopreserving the formulated harvested dopaminergic neuronal progenitor cells.

55. The method of claim 1, wherein, prior to performing the second incubation, the spheroid is dissociated to produce a cell suspension, and cells of the cell suspension are adherently cultured in the second culture vessel.

56. A therapeutic composition comprising dopaminergic neuronal progenitor cells produced using a method that comprises:
a) performing a first incubation comprising non-adherently culturing pluripotent stem cells in a first culture vessel under conditions that promote cellular spheroid formation, wherein the first incubation comprises:
i) exposing the pluripotent stem cells to a first medium that comprises an inhibitor of TGF-β/activin-Nodal signaling and an inhibitor of bone morphogenetic protein (BMP) signaling for at least one day (Day 0) in the absence of: a) an activator of Sonic Hedgehog (SHH) signaling, and b) an inhibitor of glycogen synthase kinase 3β (GSK3β) signaling; and
ii) starting on the second day (Day 1) of the first incubation, exposing the pluripotent stem cells to a second medium that comprises an activator of Sonic Hedgehog (SHH) signaling and an inhibitor of glycogen synthase kinase 3β (GSK3β) signaling; and
b) performing a second incubation comprising adherently culturing cells of the spheroid in a second culture vessel under conditions that promote differentiation into dopaminergic neuronal progenitor cells;
wherein either (i) the first incubation is performed using a microwell cell culture bag or (ii) the first incubation and the second incubation is performed using an automated cell culture system.
